(19) United States
(12) Patent Application Publication
(10) Pub. No.: US 2004/0049800 A1
(43) Pub. Date: Mar. 11, 2004

(54) Rapid methods for assessing therapeutic activity using animals expressing constitutively active G protein coupled receptors

(76) Inventors: Alan S. Kopin, Wellesley, MA (US); Martin Beinborn, Boston, MA (US)

Correspondence Address: CLARK & ELBING LLP, 101 FEDERAL STREET, BOSTON, MA, 02110, US

(21) Appl. No.: 10/458,860

(22) Filed: Jun. 11, 2003

Related U.S. Application Data
(60) Provisional application No. 60/388,450, filed on Jun. 13, 2002.

(51) Int. Cl. A01K 67/00

(52) U.S. Cl. 800/3; 800/8

(57) ABSTRACT

In general, the invention features methods that make use of animals expressing constitutively active G protein-coupled receptors for testing therapeutic efficacy and drug screening. Because these assays do not require animal breeding, they provide rapid assay results.

### CROSS REFERENCE TO RELATED APPLICATIONS

This application claims the benefit of the filing date of U.S. provisional application, U.S. S. No. 60/388,450, filed Jun. 13, 2002.

### BACKGROUND OF THE INVENTION

In general, the invention features methods for using animals expressing constitutively active G protein-coupled receptors for testing of therapeutic efficacy and drug screening.

G protein-coupled receptors form an extensive protein family with a wide variety of ligands and physiological roles. Current understanding of G protein-coupled receptor activation has, in large part, been based on the study of catecholamine receptors, such as dopamine and adrenergic receptors. The endogenous ligands of these biogenic amine receptors, together with synthetic derivatives of these small molecules, cover a spectrum of functional activities ranging from full agonists to antagonists.

Another major group of G protein-coupled receptors is activated by endogenous peptide molecules; such receptors include the mu opioid, melanocortin-4 (MC-4), pituitary adenylate cyclase activating polypeptide type I (PACAP), cholecystokinin-B/gastrin (CCK-B), and glucagon-like peptide (GLP-1) receptors. Since endogenous peptides mediate important hormone and neurotransmitter functions, there is considerable interest in whether their function can be mimicked by non-peptide drugs. This possibility is suggested by the opioid receptor system. Numerous non-peptide compounds have been identified that bind specific peptide hormone receptor subtypes with high affinity. Unlike the corresponding endogenous peptide agonists, the vast majority of these non-peptide ligands appear to lack intrinsic activity and have been pharmacologically classified as antagonists.

Because G protein-coupled receptors play an important role in human health and disease, it is important to identify synthetic agonists and antagonists for these receptors. Many currently available G protein-coupled receptor synthetic ligands are inadequate, since they lack specificity and cause adverse side effects. Thus, a need exists in the art for the identification of new G protein-coupled receptor-related therapeutics. A need also exists for a system for readily testing the efficacy of such therapeutics.

### SUMMARY OF THE INVENTION

In a first aspect, the invention generally features a method of determining whether a constitutively active G protein-coupled receptor (e.g., an orphan receptor, human receptor, dopamine (D1, D2, D2L, or D2S) receptor, mu opioid receptor, melanocortin-4 receptor, β2 adrenergic receptor, α1 adrenergic receptor, cholecystokinin-B/gastrin (CCK-BR) receptor, or glucagon-like peptide (GLP-1) receptor) has potential therapeutic activity. The method involves (a) introducing a nucleic acid encoding a constitutively active G protein-coupled receptor into a non-human animal (e.g., a vertebrate, rodent, mouse, or rat) under conditions which allow expression of the constitutively active G protein-coupled receptor in the animal in a tissue which normally expresses the receptor (e.g., neurons); and (b) without breeding the animal, assaying a phenotypic output of the expression of the constitutively active G protein-coupled receptor, whereby a positive phenotypic output relative to a control animal lacking expression of the constitutively active G protein-coupled receptor indicates that the constitutively active G protein-coupled receptor has potential therapeutic activity.

In one embodiment, the G protein coupled receptor has a peptide, lipid, small molecule, amino acid, or biogenic amine ligand. In a preferred embodiment, the constitutively active receptor is a mu opioid receptor and it includes an Asparagine at amino acid 150. In another preferred embodiment, the nucleic acid is introduced into the animal using a viral vector (e.g., an AAV vector). In yet another preferred embodiment, the constitutively active G protein coupled receptor is overexpressed in a tissue of the animal.

In a second aspect, the invention generally features a method of determining whether a G protein-coupled receptor (e.g., an orphan receptor, human receptor, dopamine (D1, D2, D2L, or D2S) receptor, mu opioid receptor, melanocortin-4 receptor, β2 adrenergic receptor, α1 adrenergic receptor, cholecystokinin-B/gastrin (CCK-BR) receptor, or glucagon-like peptide (GLP-1) receptor) is a candidate drug screening target. The method involves (a) introducing a nucleic acid encoding a constitutively active G protein-coupled receptor into a non-human animal (e.g., a vertebrate, rodent, mouse, or rat) under conditions which allow expression of the constitutively active G protein-coupled receptor in the animal in a tissue which normally expresses the receptor (e.g., neurons); and (b) without breeding the animal, assaying a phenotypic output of the expression of the constitutively active G protein-coupled receptor, whereby either a positive phenotypic output or a negative phenotypic output relative to a control animal lacking expression of the constitutively active G protein-coupled receptor indicates that the G protein-coupled receptor, or a constitutively active variant thereof, is a candidate drug screening target (e.g., an agonist, inverse agonist, or antagonist).

In one embodiment, the G protein coupled receptor has a peptide, lipid, small molecule, amino acid, or biogenic amine ligand. In another embodiment, the agonist, inverse agonist, or antagonist is a peptide, lipid, small molecule, amino acid, or biogenic amine. In a preferred embodiment, the constitutively active receptor is a mu opioid receptor and it includes an Asparagine at amino acid 150. In another preferred embodiment, the nucleic acid is introduced into the animal using a viral vector (e.g., an AAV vector). In yet another preferred embodiment, the constitutively active G protein coupled receptor is overexpressed in a tissue of the animal.

In a third aspect, the invention features a method of identifying a candidate therapeutic compound. The method involves (a) introducing a nucleic acid encoding a constitutively active G protein-coupled receptor (e.g., an orphan receptor, human receptor, dopamine (D1, D2, D2L, or D2S) receptor, mu opioid receptor, melanocortin-4 receptor, β2 adrenergic receptor, α1 adrenergic receptor, cholecystokinin-B/gastrin (CCK-BR) receptor, or glucagon-like peptide (GLP-1) receptor) into a non-human animal (e.g., a vertebrate, rodent, mouse, or rat) under conditions which allow expression of the constitutively active G protein-coupled receptor in the animal in a tissue which normally expresses the receptor (e.g., neurons); (b) without breeding the animal, assaying a phenotypic output of the expression of the constitutively active G protein-coupled receptor, whereby either a positive phenotypic output or a negative phenotypic output relative to a control animal lacking expression of the constitutively active G protein-coupled receptor indicates that the G protein-coupled receptor (e.g., an orphan receptor or human receptor), or a constitutively active variant thereof, is a drug screening target for a therapeutic compound (e.g., an agonist, inverse agonist, or antagonist); (c) contacting the G protein-coupled receptor or constitutively active variant thereof identified in step (b) with a candidate compound; and (d) measuring the activity of the G protein-coupled receptor, or constitutively active variant thereof, in the presence and in the absence of the candidate compound, whereby a candidate therapeutic compound is identified as a compound that alters the activity of the G protein-coupled receptor or constitutively active variant thereof.

In one embodiment, the agonist, inverse agonist, or antagonist is a peptide, lipid, small molecule, amino acid, or biogenic amine. In another embodiment, the G protein coupled receptor has a peptide, lipid, small molecule, amino acid, or biogenic amine ligand. In another embodiment, the agonist, inverse agonist, or antagonist is a peptide, lipid, small molecule, amino acid, or biogenic amine. In a preferred embodiment, the constitutively active receptor is a mu opioid receptor and it includes an Asparagine at amino acid 150. In another preferred embodiment, the nucleic acid is introduced into the animal using a viral vector (e.g., an AAV vector). In yet another preferred embodiment, the constitutively active G protein coupled receptor is overexpressed in a tissue of the animal.

By a “constitutively active receptor” is meant a receptor with a higher basal activity level than the corresponding wild-type receptor, or a receptor possessing the ability to spontaneously signal in the absence of activation by a positive agonist. The constitutive activity of a receptor may also be established by comparing the basal level of signaling, such as second messenger signaling, of a mutant receptor to the basal level of signaling of the wild-type receptor. A constitutively active receptor exhibits at least a 25% increase in basal activity, preferably, at least a 50% increase in basal activity, more preferably at least a 75% increase in basal level activity, and, most preferably, more than a 100% increase in basal level activity, compared to either the negative control or the wild-type receptor. It is common for a constitutively active receptor, e.g., a polymorphic constitutively active receptor, that is associated with a disease phenotype, to display a relatively small increase in constitutive activity (e.g., as little as a 25% increase). Preferably, the basal activity of a constitutively active receptor can be confirmed by its decrease in the presence of an inverse agonist.

“Basal” activity means the level of activity (e.g., activation of a specific biochemical pathway or second messenger signaling event) of a receptor in the absence of stimulation with a receptor-specific ligand (e.g., a positive agonist). Preferably, the basal activity is less than the level of ligand-stimulated activity of a wild-type receptor.

A “wild-type” receptor refers to a form or sequence of a receptor as it exists in an animal, or to a form of the receptor that is homologous to the sequence known to those skilled in the art as the “naturally-occurring” sequence. Those skilled in the art will understand “wild-type” receptor to refer to the conventionally accepted amino acid consensus sequence of the receptor with normal physiological patterns of ligand binding and signaling.

A “mutant receptor” is understood to be a form of the receptor in which one or more amino acid residues in the predominant receptor occurring in nature, e.g., a naturally occurring or wild-type receptor, have been either deleted or replaced. Alternatively additional amino acid residues have been inserted.

By “expression of said constitutively active G protein-coupled receptor” is meant transcription and translation of the receptor at a level that is at least 5%, 20% or 50% preferably, 70% or 80%, and, more preferably 90% or 100% of the wild-type level of expression in a given cell or tissue type. “Expression” also includes overexpression of the receptor, which is any level of transcription and translation that results in more than the wild-type level of receptor expression in a given cell or tissue. “Expression vectors” contain at least a promoter operably linked to the gene to be expressed.

By “therapeutic activity” is meant a level of activity sufficient to prevent, cure, stabilize, or ameliorate a condition, disease, or disorder, or some or all of its symptoms.

By a “phenotypic output” is meant any characteristic or behavior that can be detected in a non-human animal. A “positive phenotypic output” is a characteristic or behavior that correlates with a normal, healthy animal, or with the alleviation of an undesirable condition, disorder, or disease. Conversely, a “negative phenotypic output” is a characteristic or behavior indicative of an unhealthy animal or correlated with an undesirable condition, disorder, or disease.

By a “drug screening target” is meant a G protein-coupled receptor that may be used to identify a candidate therapeutic compound based on the compound's ability to alter receptor activity.

By “mu opioid receptor” is meant a polypeptide having the analgesic characteristics of the mu opioid receptor, or other associated mu opioid receptor biological activities. These activities include, for example, high affinities for analgesic and addicting opiate drugs (e.g., morphine and fentanyl) and opioid peptides (e.g., enkephalins, endorphins, and dynorphins (Rothman et al., Synapse 21:60-64 (1995); Wang et al., Proc. Natl. Acad. Sci. USA 90:10230-10234 (1993); Li et al., J. Mol. Evol. 43:179-184 (1996)). In particular examples, the mu opioid receptor has nanomolar affinities for morphine and the enkephalin analog DADLE and clear recognition of naloxonazine (Wang et al., supra; Wolozin et al., Proc. Natl. Acad. Sci. USA 78:6181-6185 (1981); Eppier et al., J. Biol. Chem. 268(35):26447-26451; Golstein et al., Mol. Pharmacol. 36:265-272 (1989)). Ligand binding initiates coupling of the mu opioid receptor to adenylate cyclase, causing a decrease in adenylate cyclase activity and a corresponding decrease in the level of intracellular cAMP (Wang et al., supra).

By “dopamine receptor” is meant a G protein-coupled receptor polypeptide that binds dopamine, dopamine analogs or agonists, has sequence and structural homology with the class A or rhodopsin family of receptors, and has the biological activities associated with a dopamine receptor. Dopamine receptors include, but are not limited to, D1, D2, D2L, D2S, D3, D4, and D5.

By “glucagon-like peptide-1 (GLP-1) receptor” is meant a G protein-coupled receptor polypeptide that binds GLP-1 and has sequence and structural homology with GLP-1 receptor subtypes and has the biological activities associated with a GLP-1 receptor. For example, the wild-type GLP-1 receptor stimulates basal and glucose-induced insulin secretion and proinsulin gene expression.

By “melanocortin-4 (MC-4) receptor” is meant a G protein-coupled receptor polypeptide that binds melanocortin.

By “β2 adrenergic receptor” is meant a G protein-coupled receptor polypeptide that binds β2 adrenergic receptor agonists and has sequence and structural homology with β2 adrenergic receptors and has the biological activities associated with a β2 adrenergic receptor.

By “α1 adrenergic receptor” is meant a G protein-coupled receptor polypeptide that binds α1 adrenergic receptor agonists and has sequence and structural homology with α1 adrenergic receptors and has the biological activities associated with an α1 adrenergic receptor.

By a “cholecystokinin-B/gastrin receptor (CCK-BR)” is meant a G protein-coupled receptor polypeptide that binds cholecystokinin polypeptide and has sequence and structural homology with CCK-BR and has the biological activities associated with CCK-BR.

For any of the receptors of the invention, the receptor utilized in the claimed assay may be derived from the animal used for the assay, or may be derived from any other animal (for example, any mammal, including humans). Alternatively, the receptor may be a synthetic receptor or an engineered receptor, so long as it possesses constitutive activity.

A “reporter construct” includes at least a promoter operably linked to a reporter gene that may be used to assay transcriptional or translational output. Such reporter genes may be detected directly (e.g., by visual inspection or detection through an instrument) or indirectly (e.g., by binding of an antibody to the reporter gene product or by reporter product-mediated induction of a second gene product). Examples of standard reporter genes include genes encoding the luciferase, green fluorescent protein, or chloramphenicol acetyl transferase gene polypeptides (see, for example, Sambrook, J. et al., Molecular Cloning: a Laboratory Manual, Cold Spring Harbor Press, N.Y., or Ausubel et al., Current Protocols in Molecular Biology, Greene Publishing Associates, New York, N.Y., V 1-3, 2000, incorporated herein by reference). Expression of the reporter gene is detectable by use of an assay that directly or indirectly measures the level or activity of the reporter gene. Preferred reporter constructs also include a response element.

A “response element” is a nucleic acid sequence that is sensitive to a particular signaling pathway, e.g., a second messenger signaling pathway, and assists in driving transcription of the reporter gene. According to the present invention, the response element may be the promoter.

By “substantially pure nucleic acid” is meant a nucleic acid (e.g., DNA or RNA) that is free of the genes which, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA which is part of a hybrid gene encoding additional polypeptide sequence.

“Transformed cell” means a cell into which (or into an ancestor of which) has been introduced, by means of recombinant DNA techniques, a DNA molecule encoding a polypeptide.

“Promoter” means a minimal sequence sufficient to direct transcription. Also included in the invention are those promoter elements which are sufficient to render promoter-dependent gene expression controllable for cell-type specific or tissue-specific regulators; or inducible by external signals or agents; such elements may be located in the 5′ or 3′ regions of the native gene. A promoter element may be positioned for expression if it is positioned adjacent to a DNA sequence so it can direct transcription of the sequence.

“Operably linked” means that a gene and a regulatory sequence(s) are connected in such a way as to permit gene expression when the appropriate molecules (e.g., transcriptional activator proteins) are bound to the regulatory sequence(s).

### BRIEF DESCRIPTION OF THE DRAWINGS

FIG. 1 (1-1 to 1-12) is a table of constitutively active Class A and Class B G protein-coupled receptors (SEQ ID NOS: 2-75). The mutations that impart constitutive activity to the receptors are indicated.

FIG. 2 is a graph showing the constitutive activity of a D146M MC-4 receptor mutant as assayed by measuring basal level cAMP production.

FIG. 3 is a graph showing the constitutive activity of the L325E CCK-BR receptor as assayed using a luciferase reporter assay.

FIG. 4 is a graph showing the sensitivity of the reporter constructs, SMS-Luc, SRE-Luc, and SRE-Luc+Gq5i to ligand-mediated activation of the mu opioid receptor.

FIG. 5 is a graph showing the constitutive activity of the Asn150Ala rat mu opioid receptor as assayed using the SRE-Luc/Gq5i luciferase reporter assay.

FIG. 6 is an illustration of a seven transmembrane domain Class I G protein-coupled receptor. Selected residues are indicated.

FIG. 7 is an illustration showing the amino acid residues conserved between the mu opioid receptor, the bradykinin B2 receptor, and the angiotensin II AT1A receptor.

FIG. 8 is an illustration showing the amino acid residues conserved between the oxytocin, vasopressin-V2, cholecystokinin-A, melanocortin-4, and α1b adrenergic receptors.

FIG. 9 is a graph showing the constitutive activity of the D146M MC-4 receptor as assayed using a luciferase reporter assay.

FIG. 10 is an illustration showing the positions relative to the CWLP motif (positions -13 and -20) conserved between the 1A adrenergic receptor, the α2C adrenergic receptor, the β2 adrenergic receptor, the serotonin 2A receptor, the cholecystokinin-B receptor, the platelet activating factor receptor, and the thyroid stimulating hormone receptor. (Conserved residues are indicated by a single letter code.)

FIG. 11 is an illustration showing a sequence alignment of the human kappa opioid receptor (ork) (SEQ ID NO: 76), the rat kappa opioid receptor (orkr) (SEQ ID NO: 77), the human mu opioid receptor (orm) (SEQ ID NO: 78), the rat mu opioid receptor (ormr) (SEQ ID NO: 79), the human delta opioid receptor (ord) (SEQ ID NO: 80), the rat type 1A angiotensin II receptor (AT1A) (SEQ ID NO: 81), and the human bradykinin receptor (B2) (SEQ ID NO: 82).

FIG. 12 is an illustration showing the amino acid sequence (top to bottom) of the mouse mu opioid receptor (SEQ ID NO: 83), the rat mu opioid receptor (SEQ ID NO: 1), the bovine mu opioid receptor (SEQ ID NO: 84), the human mu opioid receptor (SEQ ID NO: 85), the pig mu opioid receptor (SEQ ID NO: 86), the white sucker (ws) opioid receptor (SEQ ID NO: 87), the angiotensin AT-1 receptor (SEQ ID NO: 81), and the bradykinin-B2 receptor (SEQ ID NO: 82).

### DETAILED DESCRIPTION

The present invention features methods that exploit animals expressing constitutively active G protein-coupled receptors for the identification of therapeutically useful receptors, drug screening targets, and therapeutic compounds that alter G protein-coupled receptor signaling; because these methods do not require animal breeding, they provide very rapid assay results. These methods may be used, for example, for testing the therapeutic efficacy of receptors or drugs prior to or in conjunction with human clinical trials. In addition, because the present invention enables tissue-specific expression of constitutively active receptors, it also provides for assays useful for identifying new therapeutic uses for known drugs.

Constitutively Active G Protein-Coupled Receptors

Any constitutively active G protein-coupled receptor may be used to generate the animals of the invention. Such G protein-coupled receptors may recognize any ligand, for example, any peptide, lipid, small molecule, amino acid, or biogenic amine ligand. Peptide hormone receptors are particularly useful in the invention. In addition, because of the constitutive nature of the receptors, orphan receptors also represent preferred receptors for use in the assays of the invention.

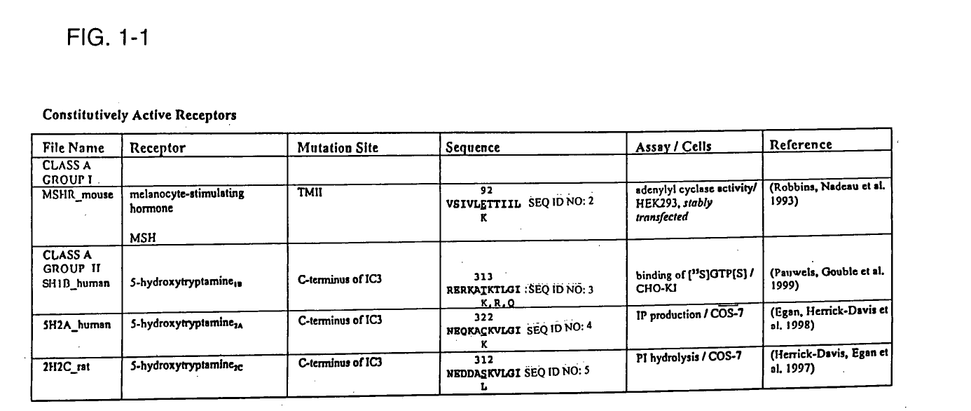

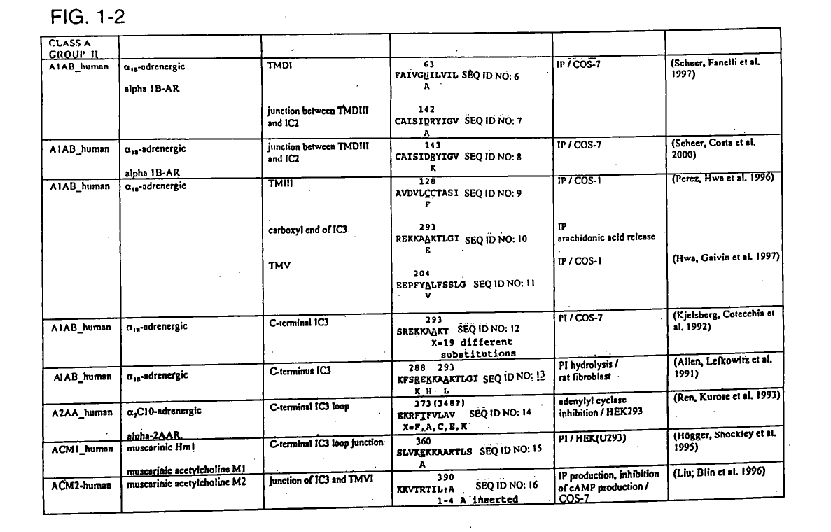

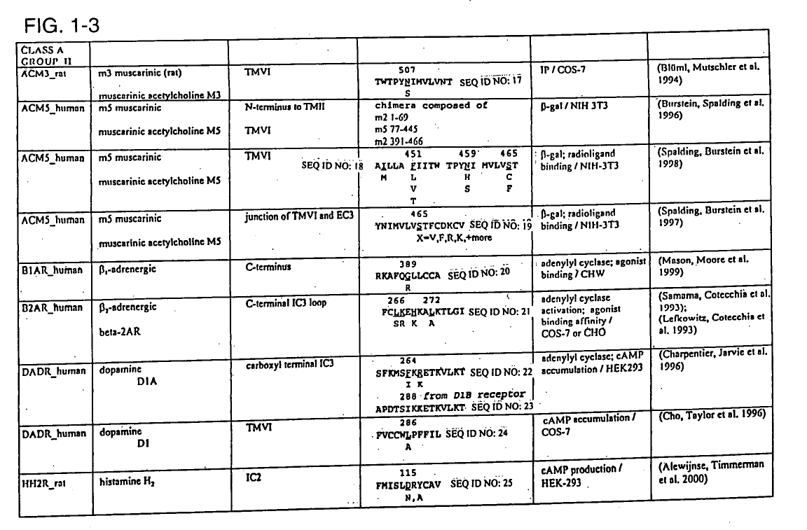

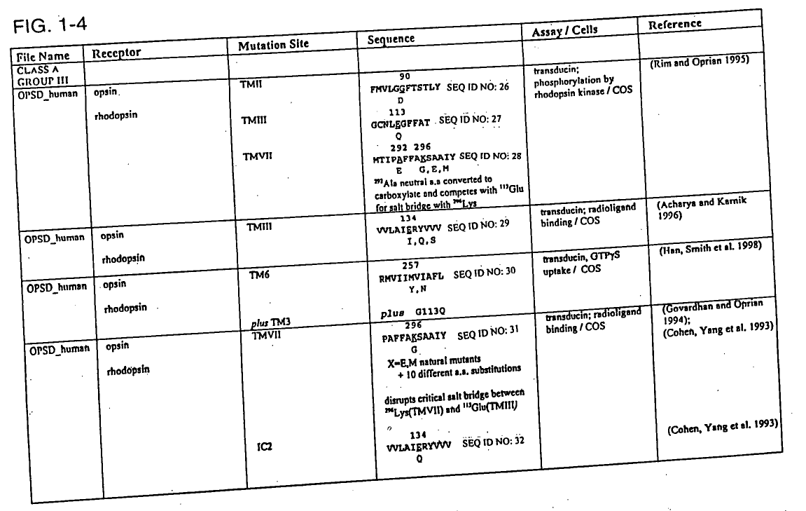

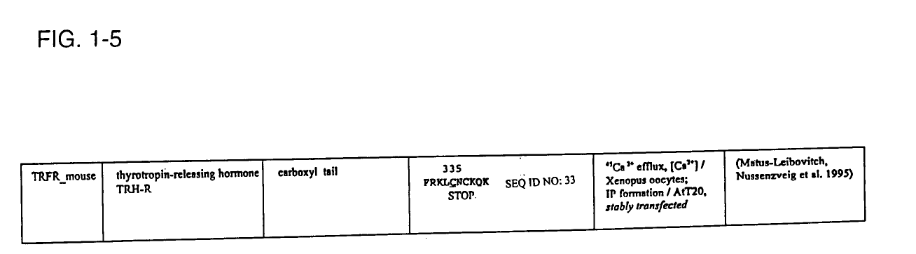

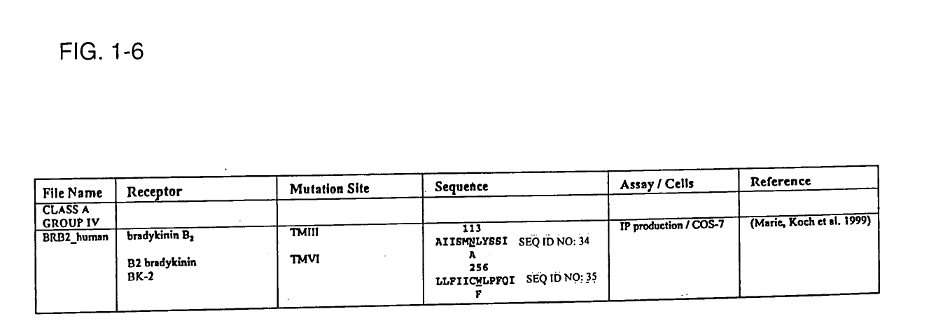

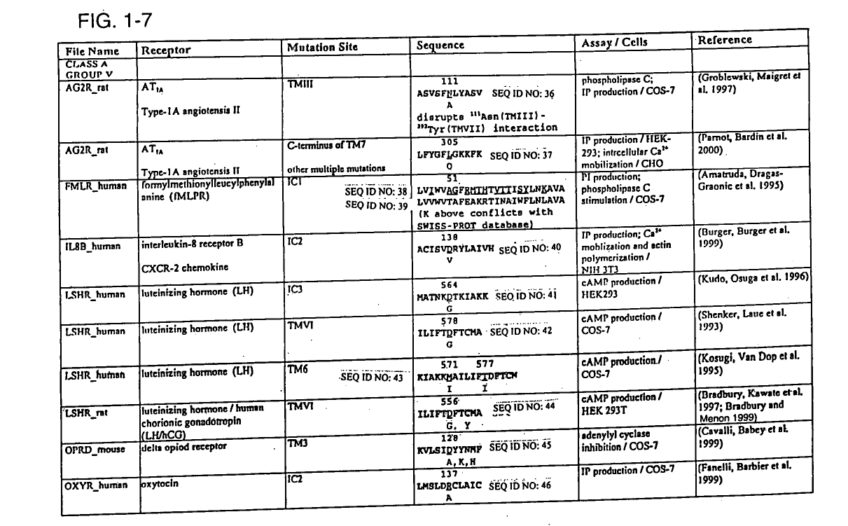

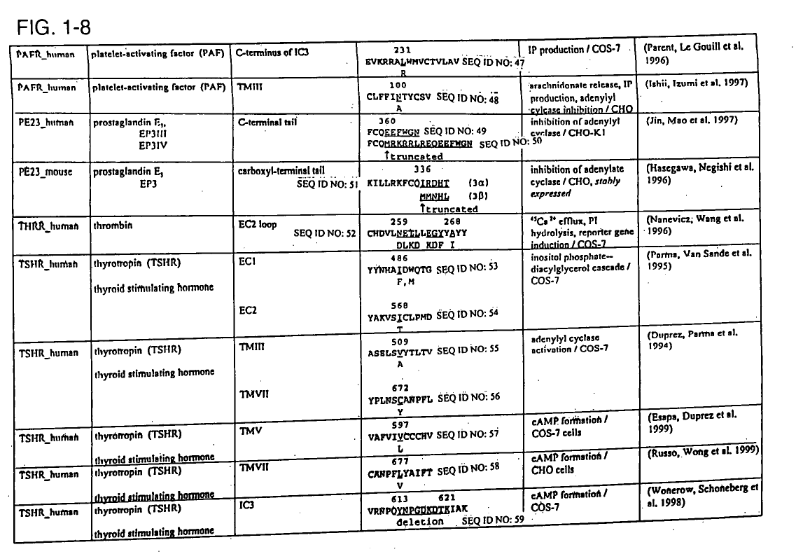

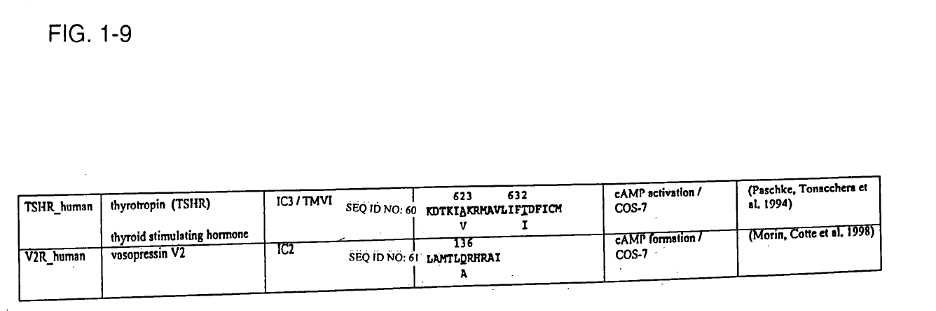

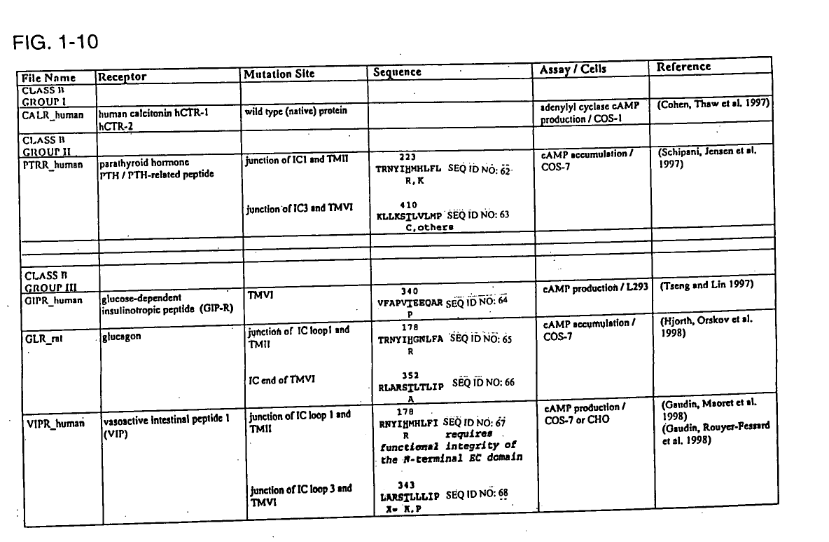

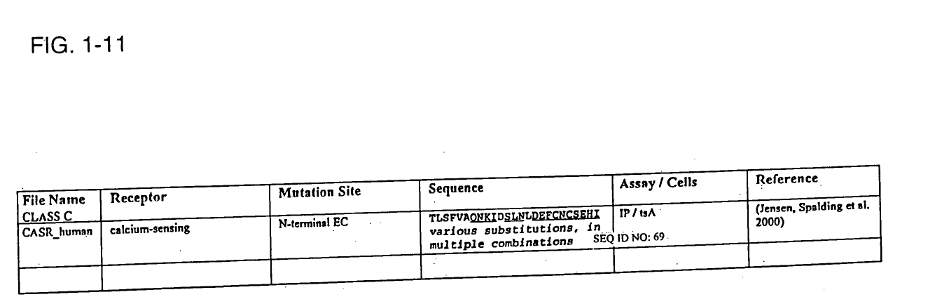

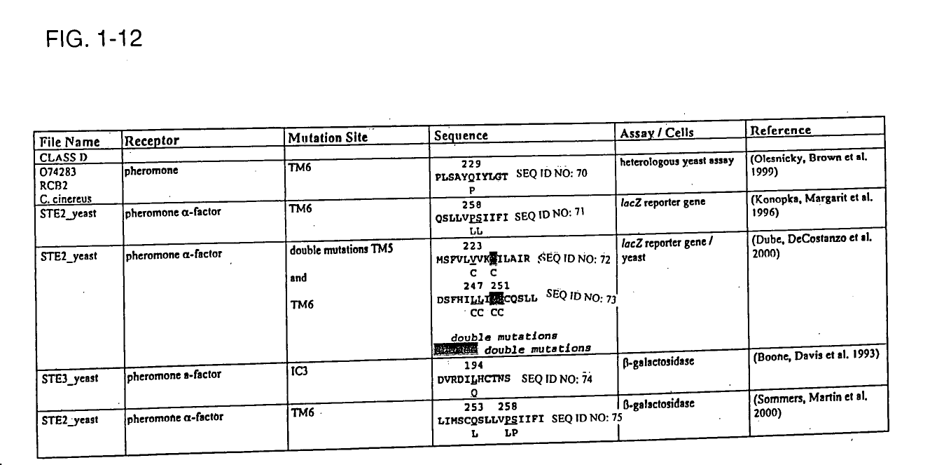

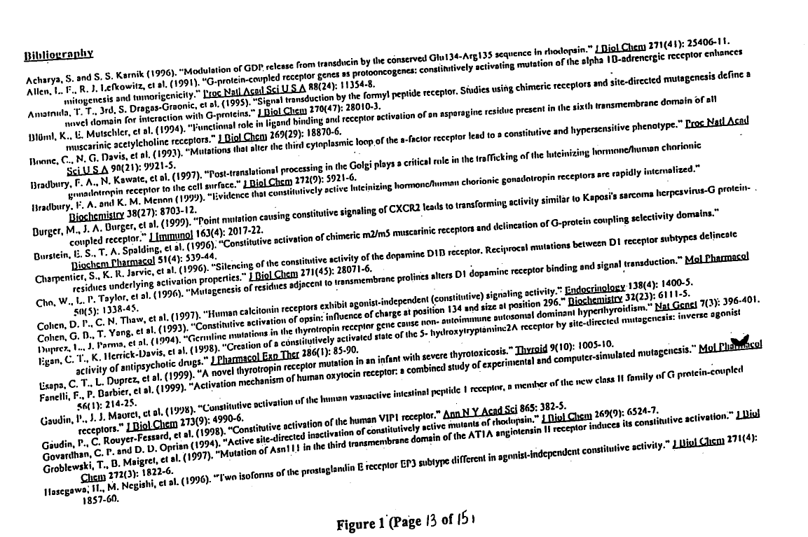

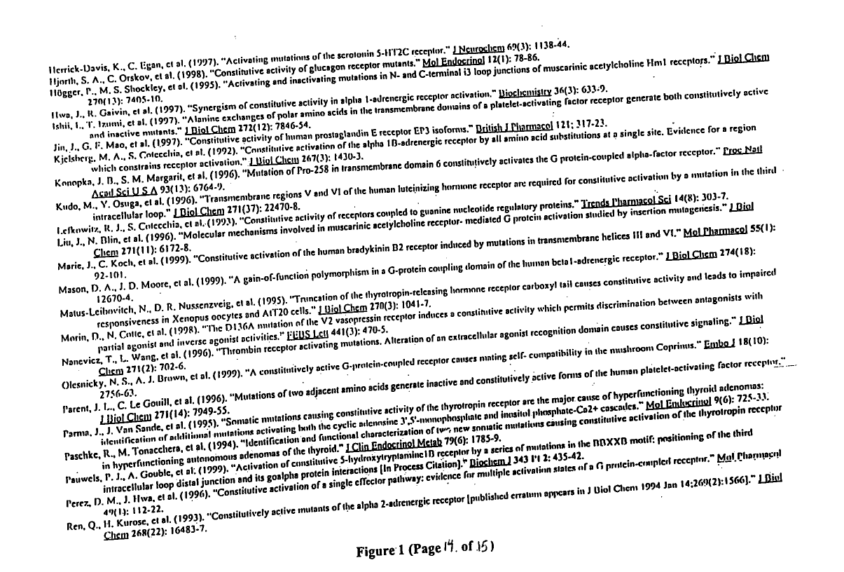

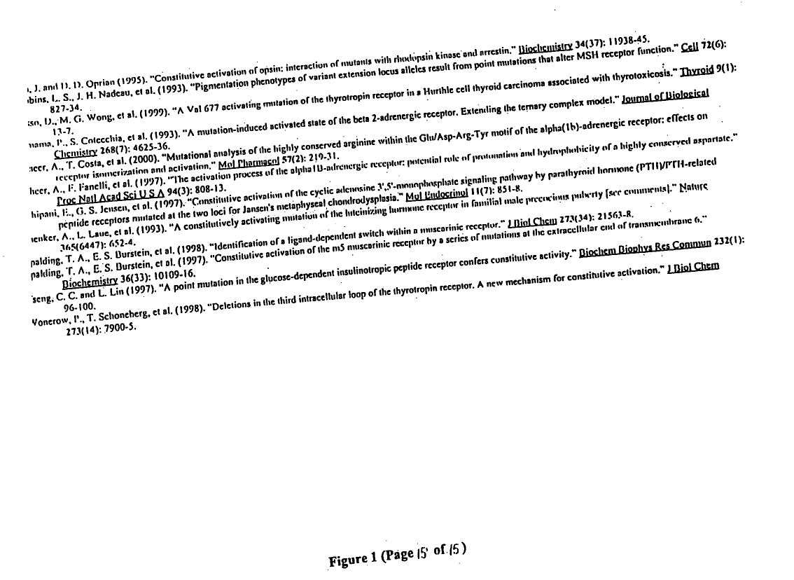

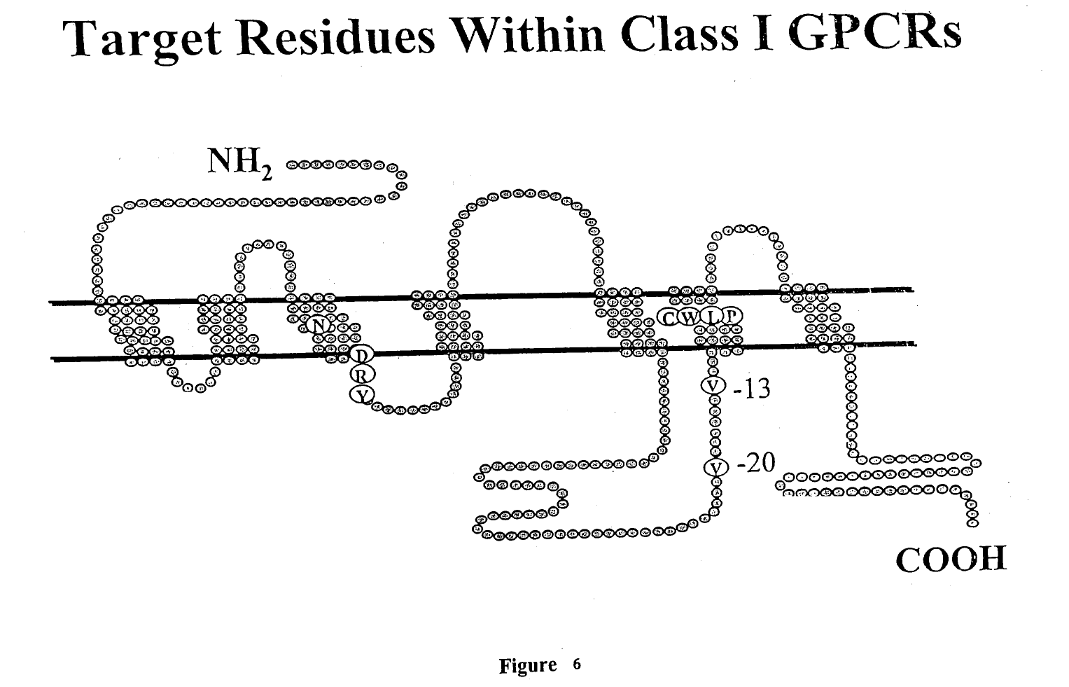

Any known wild-type or mutant G protein-coupled receptor may be exploited in the present assays. The G-protein coupled receptor may be derived from the same organism, for example, a mouse receptor for a mouse host, or may be derived from another organism, preferably a human. New constitutively active G protein-coupled receptors may also be designed for use in the invention, for example, using a database of constitutively active Class I G protein-coupled receptors (FIG. 1; FIG. 6) to target specific residues in nonconstitutively active receptors for mutation. In this approach, highly conserved regions are identified between several nonconstitutively active receptors and a number of constitutively active Class I G protein-coupled receptors in the database. This information is then used to target specific residues in the nonconstitutively active receptors for mutation. As described in detail below, targeted point mutations are introduced into the G protein-coupled receptors in this manner, which impart constitutive activity to the nonconstitutively active receptors.

To test for constitutively active receptors, receptor activity may be assayed by any method. For example, G protein-coupled receptor signaling is transduced via second messengers. By “second messenger signaling activity” refers to production of an intracellular stimulus (including, but not limited to, cAMP, cGMP, ppGpp, inositol phosphate, or calcium ions) in response to activation of the receptor, or to activation of a protein in response to receptor activation, including but not limited to a kinase, a phosphatase, or to activation or inhibition of a membrane channel. The activity of a specific G protein-coupled receptor may be determined by monitoring the level of its second messenger, for example, intracellular cAMP may be measured using a radioimmunoassay (e.g, New England Nuclear, Boston, Mass.)).

Changes in second messenger levels may also be monitored using a reporter system. Such a reporter system may include a response element that is sensitive to signaling through a particular receptor. For example, the somatostatin promoter element (SMS) is activated by coupling of receptors to either Gαq or Gαs; the serum response element (SRE) is activated by receptor coupling to Gαq; the cAMP response element (CRE) is activated by receptor coupling to Gαs and inhibited by coupling to Gαi; and the TPA response element (sensitive to phorbol esters) is activated by receptor coupling to Gαq. Each of these response elements can be employed in a reporter assay to generate a readout for activity of a specific G protein-coupled receptor.

In addition, a reporter construct for detecting receptor signaling may include a response element that is a promoter sensitive to signaling through a particular receptor. For example, the promoters of genes encoding epidermal growth factor, gastrin, or fos can be operably linked to a reporter gene for detection of G protein-coupled receptor signaling.

A wide variety of reporter constructs can be generated that are sensitive to any of a variety of signaling pathways induced by signaling through a particular receptor (e.g., a second messenger signaling pathway). For example, the elements AP-1, NF-Kb, SRF, MAP kinase, p53, c-jun, TARE can all be positioned upstream of a reporter gene to obtain reporter gene expression. Additional response elements, including promoter elements, can be found in the Stratagene catalog (PathDetect® in Vivo Signal Transduction Pathway cis-Reporting Systems Introduction Manual or PathDetect® in Vivo Signal Transduction Pathway trans-Reporting Systems Introduction Manual, Stratagene, La Jolla, Calif.).

In one embodiment, the G protein-coupled reporter assay system includes (1) a reporter construct containing a response element that is sensitive to signaling through a specific G protein, and a promoter, operably linked to a reporter gene; preferably in combination with (2) an expression vector containing a promoter operably linked to a nucleic acid encoding the receptor, wherein the receptor is coupled to a G protein or other downstream mediator to which the selected response element is sensitive. Alternatively, a G protein-coupled receptor assay includes transfection of wild-type or mutant receptors into cells followed by assessment of the levels of transcription of cell specific genes compared to the appropriate controls (e.g., transfected cells compared to nontransfected cells and the presence or absence of ligand stimulation).

The constitutively active receptors described herein make use of specific response elements that are sensitive to signaling through Gαq, Gαs, or Gαi. For example, the SMS and SRE response elements each detect an increase in basal activity of constitutively active CCK-B mutant receptor, which is coupled to Gαq. Similarly, a constitutively active rat mu opioid receptor may be assayed using a reporter construct sensitive to Gαi coupling. One response element for this assay uses the cAMP-response element (CRE), which is sensitive to Gαi mediated reductions in intracellular levels of cAMP. Signaling through the rat mu opioid receptor via Gαi inhibits adenylate cyclase, causing a decrease in intracellular cAMP. Therefore, an increase in rat mu opioid receptor signaling induces a decrease in CRE mediated reporter activity.

This reporter system may be used to identify constitutively active rat mu opioid receptors. Specifically, cells are transfected with a CRE-Luc reporter construct (Stratagene, La Jolla, Calif.) and an expression vector encoding either a wild-type or a mutant rat mu opioid receptor and stimulated with 0.5 μM or 2 μM forskolin to increase the intracellular pool of cAMP. The basal (and ligand-induced) level of receptor activity are then measured using a standard luciferase assay. Coexpression of the receptor of interest with a luciferase reporter gene construct allows one to measure light emission as a readout for basal signaling.

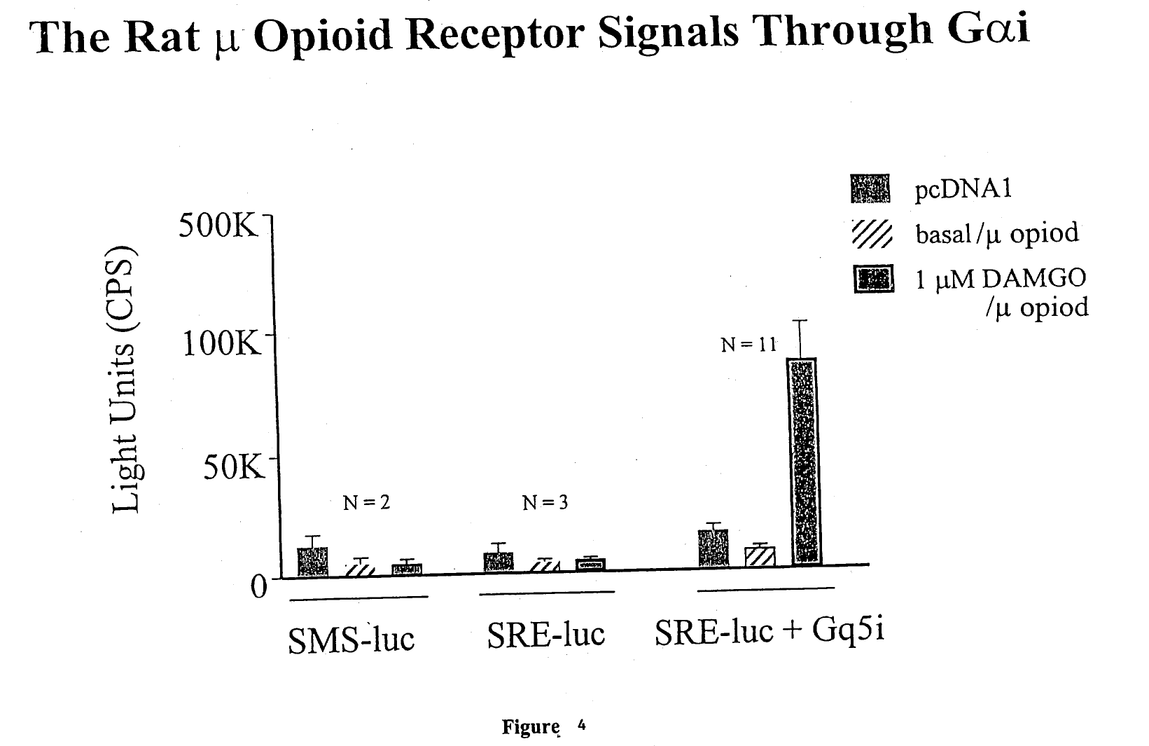

Alternatively, a positive assay for Gαi coupling (i.e., one that yields an increase in luciferase activity upon receptor activation, instead of a negative assay, one that yields a decrease in luciferase activity upon receptor activation) may be utilized (FIG. 4). Such an assay provides a detectable output signal and less interassay variation. One preferred assay system is a chimeric G protein (Gqi5, Broach and Thorner, Nature 384 (Suppl.):14-16, 1996) that contains the entire Gαq protein having five C-terminal amino acids from Gαi attached to the C-terminus of Gαq has been generated. This chimeric G protein is recognized as Gαi by Gαi coupled receptors, but switches the receptor induced signaling from Gαi to Gαq. This allows Gαi receptor coupling to be detected using a positive assay by use of the Gαq responsive SMS-Luc or SRE-Luc construct (Stratagene, La Jolla, Calif.). SMS and SRE preferably respond to Gαq mediated inositol and calcium production. Moreover, detection can be carried out in the absence of forskolin pre-stimulation of cells.

Other chimeric G proteins that can be used according to the methods of the invention include those described in Milligan, G. and S. Rees, TIPS 20:118-124, 1999, and Conklin et al., Nature 363:274-276, 1993, incorporated by reference herein. Moreover, any other chimeric G protein can be constructed by replacing or adding at least 3 amino acids, usually at least 5 amino acids, from the carboxyl terminus of a G protein (e.g., Gi, Gq, Gs, Gz, or Go) to a second G protein (e.g., Gi, Gq, Gs, Gz, or Go) which is either full-length or includes at least 50% of the amino terminal amino acids.

Expression Vectors

To generate animals according to the invention, expression vectors can be constructed using any suitable genetic engineering technique, such as those described in Sambrook et al. (Molecular Cloning: A Laboratory Manual, Cold Spring Harbor Laboratory, N.Y., (1989)). Similarly, many techniques for transfection or transformation in general are known and may be used for the expression of the constitutively active G protein-coupled receptors.

One skilled in the art will appreciate that a promoter is chosen that directs expression of the chosen gene in the tissue in which the G protein-coupled receptor is normally expressed or is desired to be expressed (see, for example, Gopalkrishnan et al., Nucleic Acids Res. 27(24):4775-4782 (1999); Huang et al., Mol. Med. 5(2):129-137 (1999)). A number of promoters are available in the art for cell-specific or tissue-specific expression. For example, any promoter that promotes expression of constitutively active dopamine receptors in neurons, preferably dopaminergic neurons, can be used in the expression constructs of the present invention. Preferred promoters for use in the invention include the β-actin and CMV promoters, which promote expression anywhere in the brain and so, for example, promote expression at brain injection sites, the neuron-specific enolase promoter, which promotes expression in neurons, and the enkephalin and substance P promoters, which promote expression in particular subsets of neurons.

One skilled in the art would also be aware that the modular nature of transcriptional regulatory elements and the absence of position-dependence of the function of some regulatory elements, such as enhancers, make modifications such as, for example, rearrangements, deletions of some elements or extraneous sequences, and insertion of heterologous elements possible. Numerous techniques are available for dissecting the regulatory elements of genes to determine their location and function. Such information can be used to direct modification of the elements, if desired. Of course an intact region of the transcriptional regulatory elements of a gene may also be used.

In certain embodiments, it may be desirable to titrate the activity of the constitutively active receptor of the invention, i.e., to decrease or reduce the level of signaling. In order to achieve this result, the constitutively active G protein-coupled receptor is expressed under the control of an inducible promoter (e.g., the tetracycline inducible promoter). Expression from the inducible promoter is regulated by a benign small molecule (e.g., tetracycline). Expression is increased or decreased by controlling the amount of the small molecule administered, or expression is turned on or off by addition or removal of the small molecule, respectively. Other inducible systems are widely available, e.g., the ecdysone inducible system (No et al., Proc. Natl. Acad. Sci, USA, 93(8):3346-3351, (1996); Invitrogen, Carlsbad, Calif.). Alternatively, it may be desirable to use a constitutive promoter to maintain a constant level and/or a high level of expression of the constitutively active receptor.

Generation of Test Animals

Animals suitable for the rapid assays of the present invention may be generated by any standard technique. In a preferred approach, animals are transduced with a viral vector (for example, an AAV vector) encoding a constitutively active G protein coupled-receptor. The G protein-coupled receptor genes may be derived from the receptor native to the transgenic organism or may be generated, for example, from a human gene and expressed in an animal under the control of an appropriate promoter.

Numerous vectors useful for this purpose are generally known and have been described (Miller, Human Gene Therapy 15:14 (1990); Friedman, Science 244:1275-1281 (1989); Eglitis and Anderson, BioTechniques 6:608-614 (1988); Tolstoshev and Anderson, Current Opinion in Biotechnology 1:55-61 (1990); Sharp, The Lancet 337:1277-1278 (1991); Cornetta et al., Nucleic Acid Research and Molecular Biology 36:311-322 (1987); Anderson, Science 226:401-409 (1984); Moen, Blood Cells 17:407-416 (1991); and Miller and Rosman, Biotechniques 7:980-990 (1989); Rosenberg et al., N. Engl. J. Med 323:370 (1990), all hereby incorporated by reference. These vectors include adenoviral vectors and adeno-associated virus-derived vectors (Burcin et al., supra; Finegold et al., supra; Vasquez et al. supra; Mannes et al. supra; Ilan et al., Seminars in Liver Disease, 19:49-59, (1999); Patijn et al., Seminars in Liver Disease 19:61-39, 1999), retroviral vectors (e.g., Moloney Murine Leukemia virus based vectors, Spleen Necrosis Virus based vectors, Friend Murine Leukemia based vectors (Ganjam, Seminars in Liver Disease, 19:27-37 (1999)), lentiviral based vectors (Human Immunodeficiency Virus based vectors etc.), papova virus based vectors (e.g., SV40 viral vectors, see e.g., Strayer et al., Seminars in Liver Disease, 19:71-81 (1999), Herpes-Virus based vectors, viral vectors that contain or display the Vesicular Stomatitis Virus G-glycoprotein Spike, Semliki-Forest virus based vectors, Hepadnavirus based vectors, and Baculovirus based vectors. Particularly preferred viral vectors are AAV vectors. Adenoviral vector delivery systems for nucleic acids encoding constitutively active G protein-coupled receptors are also useful because the adenovirus has been shown to be easily distributed to a particular site upon direct injection to that site (including neuronal sites like the intrathecal space, see Finegold et al., supra and Mannes et al. supra).

In an alternative approach, standard ex vivo viral gene transfer may be used to generate the animals of the invention. By this approach, a specific cell type or tissue is removed from an animal and genetically engineered in vitro using viral gene transfer vectors. The genetically engineered cell or tissue is subsequently returned to the animal. In this type of gene transfer protocol, highly infectious viral vectors with broad tropisms, such as those with amphotropic envelope glycoprotein are particularly useful, (e.g., glycoprotein of the Moloney murine leukemia virus or glycoprotein G of the vesicular stomatitis virus (VSVG)). For example, in one embodiment, a constitutively active G protein-coupled receptor of the present invention is administered to an animal using ex vivo gene delivery by (i) transfecting a selected cell type in vitro with nucleic acid encoding the selected receptor; (ii) allowing the cells to express the receptor; and (iii) administering the modified cells to the animal to allow the expression of the encoded constitutively active G protein-coupled receptor.

In another approach, delivery of a viral vector encoding a constitutively active G protein-coupled receptor may be achieved by means of an accelerated particle gene transfer gun. The technique of accelerated particle gene delivery is based on the coating of nucleic acid to be delivered into cells onto extremely small carrier particles, which are designed to be small in relation to the cells sought to be transformed by the process. The nucleic acid encoding the desired gene sequence may be simply dried onto a small inert particle. The particle may be made of any inert material such as an inert metal (gold, silver, platinum, tungsten, etc.) or inert plastic (polystyrene, polypropylene, polycarbonate, etc.). Preferably, the particle is made of gold, platinum or tungsten. Most preferably the particle is made of gold. Gene guns are commercially available and well known in the art, for example, see U.S. Pat. No. 4,949,050; U.S. Pat. No. 5,120,657 (available from PowderJect Vaccines, Inc. Madison Wis.); or U.S. Pat. No. 5,149,655.

Alternatively, the viral expression vectors can be administered directly by any of a variety of routes including by intravenous, (IV), intramuscular (IM), intraperitoneal (IP), and subcutaneous administration. The G protein-coupled receptor-expressing vector may also be administered to mucosal surfaces by, for example, the nasal or oral (intragastric) route.

Animals suitable for the assays of the present invention may be obtained from standard commercial sources such as Taconic (Germantown, N.Y.).

The present invention provides animals expressing constitutively active G protein-coupled receptors. In addition, these animals may also express a G protein-coupled reporter system including a reporter contruct containing a response element that is sensitive to signaling through a specific G protein, and a promoter, operably linked to a reporter gene. Exemplary reporter constructs are described above and are available in the art. If reporter assays are exploited, animals carrying those reporter genes are utilized; constitutively active G-protein coupled receptor expression vectors are then introduced into the animals, and reporter expression is assayed in the absence of animal breeding.

Animal Screening Assays

The present invention provides screening assays that are particularly rapid because they do not require animal breeding. The animals expressing constitutively active G protein-coupled receptors may be used for identifying new therapeutic compounds or testing the therapeutic efficacy by any reporter or behavioral assay for receptor function.

For example, animals may express a G protein-coupled reporter system including a reporter construct containing a response element that is sensitive to signaling through a specific G protein, and a promoter, operably linked to a reporter gene. Constitutively active G protein-coupled receptor expression vectors are introduced into these animals, and the reporter systems are employed in sensitive screens for testing therapeutic compounds that modulate receptor activity.

Alternatively, behavioral assays may be used to monitor phenotypic output and thereby identify constitutively active G protein-coupled receptors having therapeutic activity or therapeutic compounds that modulate G protein-coupled receptor activity. A number of behavioral assays are available in the art (see, for example, Crawley, What's Wrong with My Mouse?, Behavioral Phenotyping of Transgenic and Knockout Mice, John Wiley & Sons, Inc., New York; Crawley et al., Current Protocols in Neuroscience, John Wiley & Sons, Inc., New York; and Enna et al., Current Protocols in Pharmacology, John Wiley & Sons, Inc., New York). In one particular example, animals in which a constitutively active mu opioid receptor is expressed are expected to have a decreased sensitivity to pain in the tail flick response to radiant heat (the amount of time it takes for the rat to remove its tail from a heat source); a therapeutic compound that activates the mu opioid receptor further decreases this pain sensitivity and can be identified using the assay.

The assays described herein are useful for identifying receptors or compounds as new therapeutics, or can be utilized for identifying a G protein-coupled receptor as a useful drug target. Alternatively, these assays may be utilized for testing the therapeutic efficacy of new or known candidate drugs. In addition, the present methods may be used to identify new thereapeutic uses for known drugs. The present assays are particularly useful when carried out prior to, or in conjunction with, human clinical trials.

### EXAMPLE 1

### Constitutively Active Mu Opioid Receptor

This example describes the identification of novel constitutively active rat mu opioid receptors and the use of nucleic acids encoding these receptors to generate animals useful in drug-screening or testing of therapeutic efficacy of receptors or compounds.

Identifying Regions of Homology in the Mu Opioid Receptor

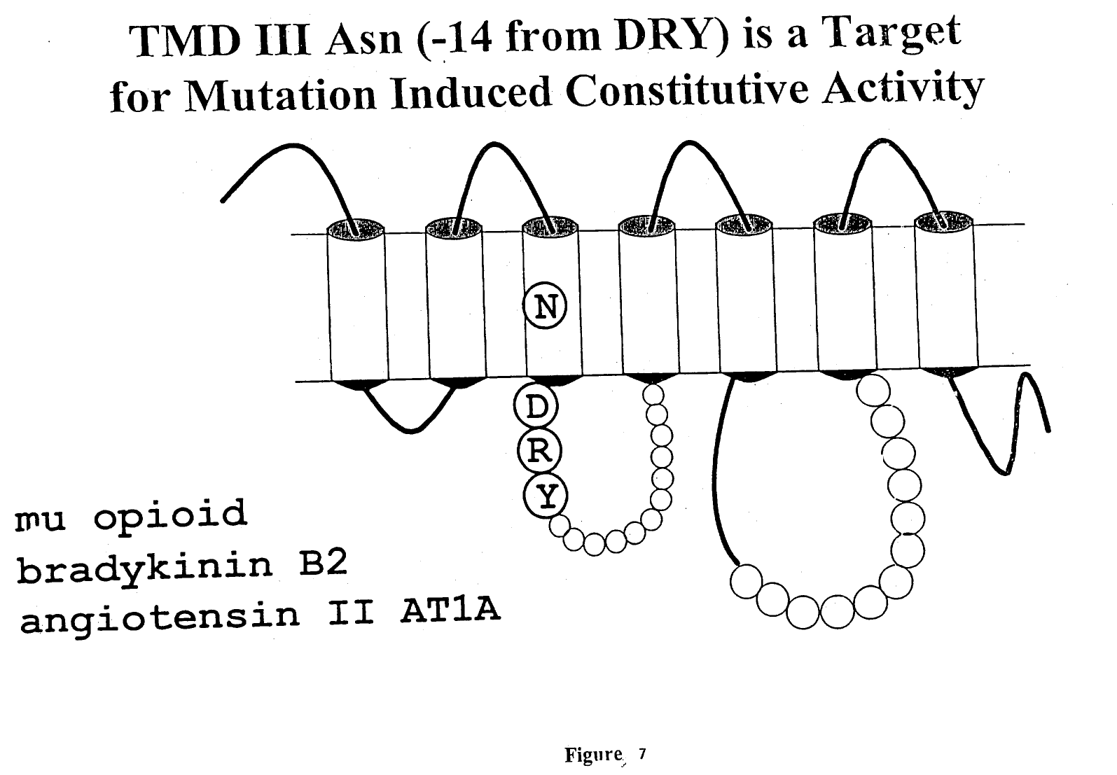

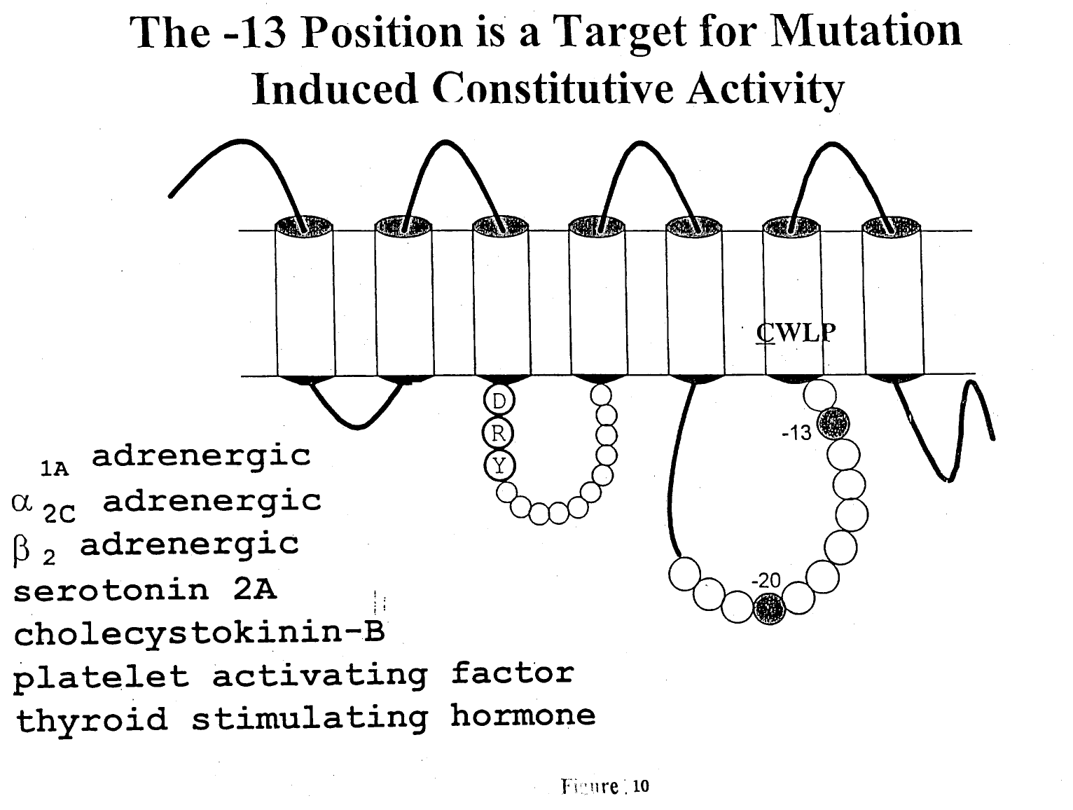

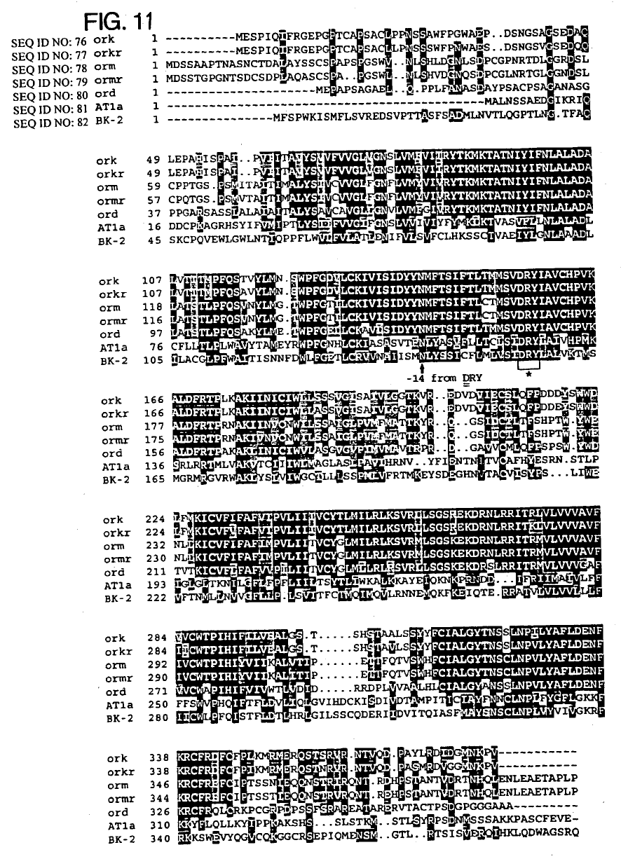

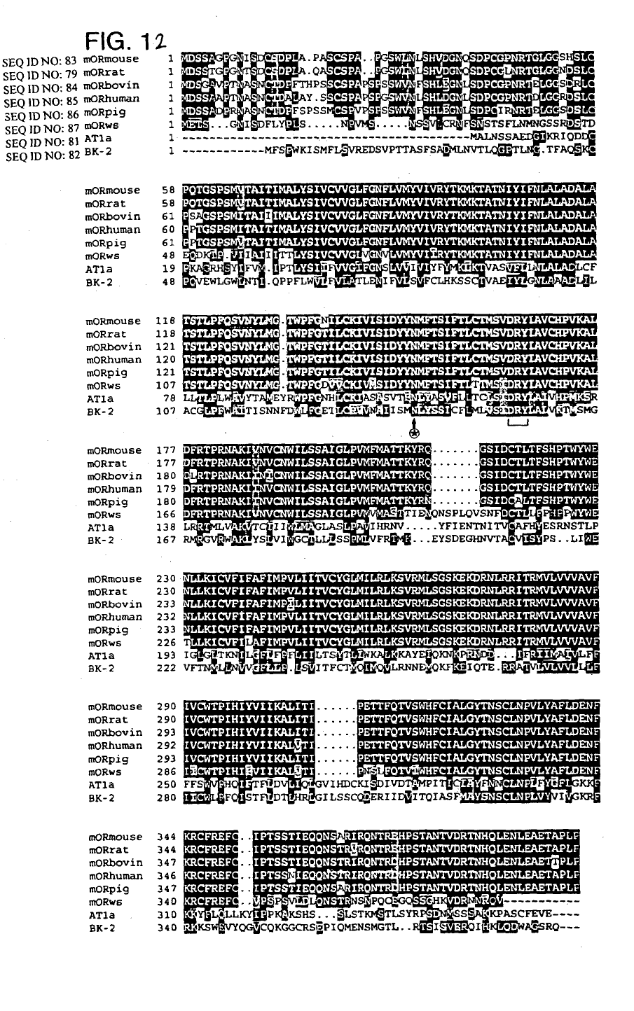

A database containing sequence information for known constitutively active Class A G protein-coupled receptors was generated by compiling available information from the prior art (see FIG. 1). The database was then used to identify key residues within Class A G protein-coupled receptors that are important for constitutive activity. These highly conserved residues are illustrated in FIG. 8. Of particular interest was the Asn residue at position 150 of SEQ ID NO: 1 in transmembrane domain III, which is conserved between the rat mu opioid receptor, the bradykinin B2 receptor, and the angiotensin II AT1A receptor (see FIG. 7; FIG. 11; FIG. 12). The ‘DRY’ motif at position 164-166 of SEQ ID NO: 1 is conserved between the oxytocin receptor, the vasopressin-V2 receptor, the cholecystokinin-A (CCK-A) receptor, the melanocortin-4 (MC-4) receptor, and the α_{1B} adrenergic receptor (see FIG. 8). It is important to note that this general motif, although not necessarily consisting of the specific residues ‘DRY’ (an alternative is, e.g., ‘ERY’), is conserved among all class A G protein-coupled receptors. In addition, the position corresponding to 13 residues N-terminal to the ‘CWLP’ motif is functionally conserved between the 1A adrenergic receptor, the α2C adrenergic receptor, the β2 adrenergic receptor, the CCK-B receptor, the platelet activating factor receptor, and the thyroid stimulating hormone receptor (see FIG. 10) in that mutation of the amino acid at position -13 in each of these receptors results in constitutive activity. “Functionally conserved” means that the same amino acids are not necessarily present, but mutations in homologous or surrounding positions can result in constitutive activity.

Generating Mutant Mu Opioid Receptors

Based on the homology between the mu opioid receptor, the bradykinin B2, and the angiotensin II AT1A receptors at the Asn residue at position 150 of SEQ ID NO: 1, we chose to generate a rat mu opioid receptor having a point mutation at this position. An Asn150Ala mutation was introduced into the rat mu opioid receptor using standard molecular biological techniques. This mutant gene was then subcloned into expression vector pcDNA1 (Sambrook et al. supra). Other constitutively active mu opioid receptors may be generated using this or any other technique.

Assaying Mutant Mu Opioid Receptors for Constitutive Activity

Reagents & Solutions: The cell culture media used in the assays described below was Gibco BRL # 12100-046. This media was made according to manufacturer's recipe, pH adjusted to 7.2, filtered (0.22 micron pore), and supplemented with 1% Pen/Strep (Gibco #15140-122; 100% penicillin G 10,000 units/ml, and streptomycin 10,000 μg/ml) and 10% fetal bovine serum. Cell culture media lacking 10% fetal bovine serum was also made. DNA used in the transfection experiments was purified and quantitated by measuring the absorbance at OD₂₆₀. A LucLite Luciferase Assay Kit (Packard) was used to quantitate luciferase activity. Transfections were carried out using LipofectAMINE Reagent (Gibco #18324-012).

Constitutive activity of the Asn150Ala mutant rat mu opioid receptor was assessed using a luciferase assay. The rat mu opioid receptor is a Gαi coupled receptor. Therefore we chose to use the Gq5i reporter system, described in detail above (Broach and Thorner, supra), which switches the signaling pathway from Gαi to Gαq for reliable positive readout. HEK293 cells were transfected with the reporter construct SRE-Luc, an expression vector containing nucleic acid encoding Gq5i (Broach and Thorner, supra), and an expression vector containing nucleic acid encoding either the wild-type or the Asn150Ala mutant rat mu opioid receptor. Basal and ligand-stimulated luciferase activity was measured. The ligand used in this assay was [D-Ala²-MePhe⁴, Gly-ol⁵]enkephalin] (DAMGO). As a negative control, HEK293 cells were transfected with pcDNA1 (empty vector DNA), SRE-Luc, and the expression vector containing nucleic acid encoding Gq5i (Broach and Thorner, supra).

The luciferase assay was carried out as follows. On day 1, HEK293 cells in a T75 flask were washed with 15 ml serum-free media (or PBS), trypsinized with 5 ml 0.05% trypsin-EDTA (Gibco #25300-062), incubated at 37° C. for 3 minutes at which time 6-7 ml complete HEK293 media (Gibco #12100-046) and 10% Fetal Bovine Serum (Intergen #1050-90) were added. Thereafter, cells were collected in 50 ml centrifuge tubes, pelleted at 800-900 rpm (RCF ˜275), and resuspend in 20 ml complete media. The cells were counted using a haemocytometer and diluted to 85,000 cells/ml in complete media. Using a repeat pipettor or cell plater, 100 μl of cells were added to each well of a Primaria 96-well plate (Falcon #353872). Cells were then incubated at 37° C., 5% CO₂ until use at 48 hours.

On day 3, cells were transfected using LipofectAMINE™ according to the manufacturer's protocol (Gibco #18324-012, Rockville, Md.).

On day 4, cells were stimulated as follows. Ligands for the receptor, either DAMGO or a non-peptide ligand (e.g., naloxone or naltrexone), were diluted to a desired concentration in serum-free media containing 0.15 mM PMSF (or other protease inhibitor(s)). The transfection media was then completely removed from cells and 50-100 μl stimulation media (i.e., media containing candidate ligands or the corresponding ligand free solvent) was added to each well. The cells were incubated for the desired time (standard is overnight) at 37° C., 5% CO₂, although the optimal stimulation time may vary depending on the particular receptor used. The optimal incubation time may be determined systematically by testing a range of incubation times and determining which one yields the highest level of stimulation. For concomitant assessment of two ligands (e.g., ligand induced inhibition of forskolin stimulated CRE activity) each stimulus is prepared at two times the desired final concentration and mixed in equal volumes prior to addition to cells.

On day 5, an assay for luciferase expression was carried out according to the manufacturer's instructions (Packard, Meridin, Conn.)

Results: Mu Opioid Receptor

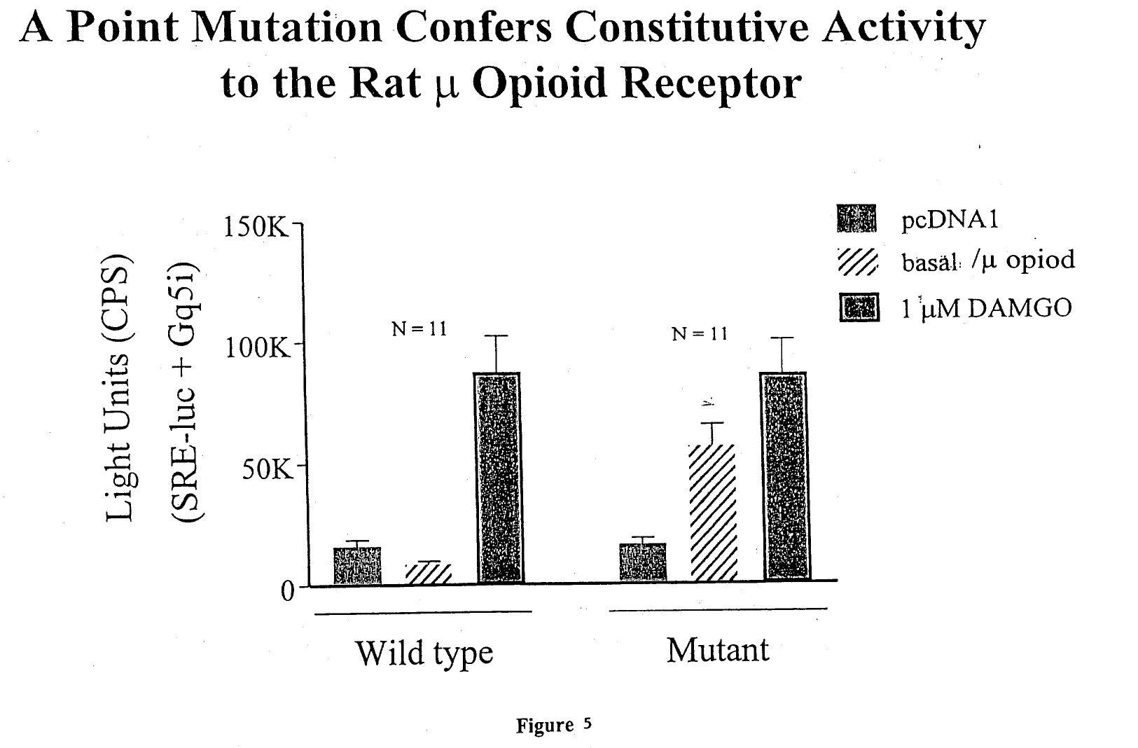

Mutation of the Asn residue at position 150 of SEQ ID NO: 1 to Ala yielded a constitutively active rat mu opioid receptor. In FIG. 5 and Table 1, below, the results of the wild-type and Asn150Ala mutant rat mu opioid receptors are compared side by side. Shown in FIG. 5 are the basal and ligand-stimulated activities of the wild-type rat mu opioid receptor and the basal activity of the negative control vector (pcDNA 1 lacking any encoded gene). The basal activity of the wild-type rat mu opioid receptor is exceeded by the basal activity of the negative control vector. There is a significant increase (approximately 6.5 fold) in basal activity of the Asn150Ala mutant mu opioid receptor, indicating that the mutant mu opioid receptor is constitutively active.

TABLE 1

| | Basal Activity | Ligand Induced Activity |
|---|---|---|
| Receptor | (Light Emission) | (Light Emission) |
| pcDNA 1 | 16,041 | 16,746 |
| (SRE + Gq5i) | | |
| wild-type rat mu opioid | 8,436 | 87,461 |
| receptor | | |
| (SRE + Gq5i) | | |
| Asn150Ala rat mu opioid | *56,498 | 86,996 |
| receptor | | |
| (SRE + Gq5i) | | |

*6.5-fold stimulation of basal level activity.

Constitutively Active Mu Opioid Receptor Animals

In a preferred approach, a construct is generated encoding the constitutively active (Asn150Ala) rat mu opioid receptor, or an equivalent mutant receptor from another organism, in a vector suitable for expression in the neurons of an animal. Exemplary promoters for neuron expression include, without limitation, the β-actin, CMV, neuron-specific enolase, enkephalin, and substance P receptors. Such expression constructs are introduced into animals through techniques well known to the skilled artisan, and described herein. In addition to the constitutively active G protein-coupled receptor, the animal may also express a reporter system sensitive to G protein-coupled receptor activity. Examples of such reporter systems are provided herein.

The effect of a test compound on G protein-coupled receptor activity is then assayed in the animal. Reporter assays for G protein-coupled receptor signaling are well known in to the art, and examples of such assays are described herein.

Alternatively, behavioral or drug response assays may be used. Any appropriate assay for pain response may be utilized (see, for example, Crawley, What's Wrong with My Mouse?, Behavioral Phenotying of Transgenic and Knockout Mice, John Wiley & Sons, Inc., New York; Crawley et al., Current Protocols in Neuroscience, John Wiley & Sons, Inc., New York; and Enna et al., Current Protocols in Pharmacology, John Wiley & Sons, Inc., New York). In one particular example, the effect of a constitutively active mu opioid receptor or a test compound on mu opioid receptor signaling in rodents can be assayed using a tail flick experiment, as described in Pollack et al. (Pharm. Res. 17(6):749-53, 2000). The tail flick response to radiant heat (the amount of time it takes for the rat to remove its tail from a heat source) determines the analgesic effect of the constitutively active mu opioid receptor or a compound acting at the receptor. Animals can be separated into a test group, which receives a test compound, and a control group, which does not receive the test compound. The responses of the two groups can be compared by the tail flick assay. Reduced sensitivity of a rat tail to heat is considered a phenotypic output characteristic of therapeutic activity and identifies the constitutively active receptor as having such therapeutic activity. This phenotypic output also identifies useful therapeutic compounds. Such therapeutic compounds may be newly discovered drugs and/or compounds or proteins being tested for therapeutic efficacy, for example, prior to or in conduction with clinical trials.

### EXAMPLE 2

### Constitutively Active Dopamine Receptors

This example describes methods for the identification of novel constitutively active dopamine receptors and the use of nucleic acids encoding these receptors to generate animals useful in drug-screening or testing of therapeutic efficacy of receptors or compounds.

Mammalian dopamine receptors are seven transmembrane domain G protein-coupled proteins that fall into the class A or rhodopsin family based on conservation of amino acid sequence. Dopamine receptors can be further divided into two major types, D1-like and D2-like. These receptor groups are distinguished based on gene structure, signal transduction pathways, and sensitivity to class specific agonist and antagonist drugs (Emilien et al., Pharmacol. Ther. 84:133-156 (1999); Missale et al., Physiol. Rev. 78:189-225 (1998); Vallone et al., Neurosci. Biobehav. Rev. 24:125-132 (2000). The D1-like receptors include the D1 and D5 subtypes. These receptors are encoded by a single exon and signal primarily through Gs mediated activation of adenylate cyclase. The D2-like receptors include the D2, D3, and D4 subtypes. Each of the D2-like receptors is encoded by multiple exons offering the potential for alternatively spliced variants to exist. Dopamine-mediated signaling through the D2-like receptors is primarily through Gi/o induced inhibition of adenylate cyclase and modulation of ion channels.

The predominant dopamine receptors found in the striatum are the D1 and D2 subtypes (Emilien et al., Pharmacol. Ther. 84:133-156 (1999). Expression has been shown by in situ hybridization, immunohistochemistry, and receptor autoradiography. Although it is agreed that the D1 and D2 receptors are highly expressed in striatum, the degree to which there is coexpression of D1 and D2 receptors within individual striatal neurons remains controversial (Missale et al., Physiol. Rev. 78:189-225 (1998); Surmeier et al., J. Neurosci. 16:6579-6591 (1996); Aizman et al., Nat. Neurosci. 3:226-230 (2000). Many studies have suggested that D1 receptors are expressed on dynorphin/substance P neurons whereas D2 receptors appear preferentially expressed on enkephalin-producing cells. Others, using confocal microscopy and functional readouts (e.g. sodium channel activation), suggest there is coexpression of both the D1 and D2 receptors in many, if not all, striatal neurons.

It is likely that both striatal D1 and D2 receptors modulate locomotor function, and both are therefore useful targets for the development of therapeutics for Parkinson's disease. Parkinson's disease affects about 1% of adults over age 60. The full clinical manifestations of Parkinson's disease include bradykinesia, rigidity, tremor, and gait abnormalities. The disease results from degeneration of the dopaminergic nigrostriatal pathway. The trigger for the degenerative process in most cases remains unknown. A minority of cases results from genetic abnormalities (e.g. mutation in the alpha synuclein or the Parkin gene) (Rohan de Silva et al., Current Opinion in Genetics & Development 10:292-298 (2000). With the gradual loss of dopaminergic neurons in the substantia nigra, there is progressive damage to the axonal projections that innervate the striatum. The loss of nigrostriatal dopaminergic neurons leads to a decrease in dopamine mediated striatal signaling (Rohan de Silva et al., Current Opinion in Genetics & Development 10:292-298 (2000); Emilien et al., Pharmacol. Ther. 84:133-156 (1999); Missale et al., Physiol. Rev. 78:189-225 (1998)). In humans as well as in rodents and nonhuman primates, toxins that destroy dopaminergic neurons (e.g. MPTP, 6-OH dopamine) result in the acute onset of Parkinsonian symptoms. Use of these toxins has enabled the development of animal models of Parkinson's disease.

Therapeutic strategies for Parkinson's disease are aimed at restoring dopaminergic activity in the striatum. One means to achieve this is to increase central dopamine levels. Levo-dopa (L-dopa), the precursor of dopamine, has been the primary drug used for this purpose. When administered peripherally, L-dopa (unlike dopamine) crosses the blood brain barrier and is then enzymatically converted to dopamine. In patients with Parkinson's disease, loss of nigrostriatal presynaptic cells leads to dopamine depletion despite intact striatal postsynaptic neurons. With disease progression pharmacotherapy is ultimately insufficient to restore normal striatal dopaminergic signaling. In addition, L-dopa administration to patients with advanced Parkinson's disease results in dyskinesias and periods of marked fluctuation in motor activity (‘on-off effect’). Alleviation of these side effects has been a major challenge in the treatment of Parkinson's disease and has prompted a search for therapeutic strategies that can provide a sustained level of dopaminergic signaling.

In the present invention, constitutively active dopamine receptors are expressed in animals and used as novel and sensitive tools for identifying therapeutic receptors or compounds and assaying the therapeutic efficacy of receptors or compounds useful in the treatment of Parkinson's Disease, as well as in other disorders of dopaminergic neurons.

Constitutively Active Dopamine Receptors

It is well established that the D1 receptor is coupled to Gs mediated activation of adenylate cyclase, which in turn leads to an elevation of cellular cAMP. D1 receptor activation of Gs was confirmed using both the luciferase assay described herein as well as a cAMP radioimmunoassay. In contrast, D2 receptors (both long and short isoforms) are linked to Gi/o coupled pathways. Activation of the D2 receptor leads to alpha subunit-mediated inhibition of adenylate cyclase with a resultant decrease in cAMP (Emilien et al., Pharmacol. Ther. 84:133-156 (1999); Missale et al., Physiol. Rev. 78:189-225 (1998); Vallone et al., Neurosci. Biobehav. Rev. 24:125-132 (2000). Activation of Gi/o was also confirmed for the D2L and D2S receptors by expressing these receptors Gi/o in HEK293 cells and measuring activity with the Gq5i/SRE luciferase reporter gene assay described above.

In addition to these major pathways, there is evidence that second messenger signaling linked to dopamine receptors includes certain other pathways that are highly cell type specific (Missale et al., Physiol. Rev. 78:189-225 (1998); Jiang et al., Proc. Natl. Acad. Sci. USA 98:3577-3582 (2001). Stimulation of dopamine receptors potentially results in activation of potassium channels, inhibition of calcium currents, and activation of mitogen activated protein kinase. In addition, in certain cellular milieus, both the D1 and D2 receptors have been shown to activate phospholipase C, leading to phosphatidylinositol-mediated increases in intracellular calcium.

Assays based on any of the above signaling pathways may be used to identify or confirm constitutive activity for a dopamine receptor simply by looking for increased activity relative to a wild-type control receptor, as described herein.

In an exemplary approach, to isolate constitutive dopamine receptors, the relevant dopamine receptor cDNAs (e.g., D1, D2S, or D2L) are obtained or generated by PCR and preferably cloned into the expression vector, pcDNA1.1. Single stranded uracil template is then preferably used as the template for site-specific mutagenesis by standard techniques.

Potential amino acid targets for mutagenesis include two D1 receptor (Cho et al., Mol. Pharmacol. 50:1338-1345 (1996); Charpentier et al., J. Biol. Chem. 271:28071-28076 (1996)) and one D2 receptor (Wilson et al., J. Neurochem. 77:493-504 (2001)) point mutations reported to confer ligand independent signaling to the respective receptor. These may be generated as previously described (Beinborn et al., Nature 362:348-350 (1993); Kopin et al., J. Biol. Chem. 270:5019-5023 (1995)) and assessed by any of the assays described herein. These mutations, as characterized in the literature, confer only a minimal level of constitutive activity. Ideally, a basal level of signaling can be achieved which approximates >50% of the dopamine-stimulated maximum activity. To enhance activity, serial amino acid substitutions may be introduced in candidate locations. This approach produces receptors with a wide range of basal signaling including ones with marked constitutive activity (Kjelsberg et al., J. Biol. Chem. 267:1430-1433 (1992); Scheer et al., Proc. Natl. Acad. Sci. USA 94:808-813 (1997). An additional strategy, which may be used, is to introduce combinations of weakly activating mutations in an attempt to further increase basal signaling. Specific mutations that may be introduced into the D1 receptor include replacement in intracellular loop 3 of the amino acid -20 from the “CWLP” sequence with either an I, E, or S, or replacement in transmembrane region 6 of the L in the “CWLP” sequence with either an A, V, K, or E. Specific mutations that may be introduced into the D2 receptor include replacement in intracellular loop 3 of the amino acid -13 from the “CWLP” sequence with either an E, K, R, A, S, or C.

In addition, the deduced amino acid sequence of the D1 and D2 receptors includes “hotspots” relative to conserved signature motifs (e.g., DRY) in other class A G protein-coupled receptors. Additional mutants may be constructed based on this hotspot in intracellular loop II. For example, the D in the “DRY” sequence may be replaced with either an M, T, V, I, or A, or the R may be replaced with either an A or K. As above, these receptors are generated by site-specific mutagenesis, sequenced for confirmation of the amino acid alteration, and screened for constitutive activity. Agonist induced signaling is included as a positive control; this also enables normalization/comparison of elevations in basal signaling (i.e. agonist induced signaling=100%).

In the alternative, random mutations may be introduced into a limited domain of the dopamine receptor of interest; mutant receptors are then screened for ligand independent signaling. Preferred domains for such mutagenesis include the amino and carboxy ends of the third intracellular loop as well as the sixth transmembrane domain.

As described above, mutants may be screened with a series of luciferase reporter gene assays to detect Gs, Gi/o, and Gq mediated signaling. To confirm that Gs coupled mutants are constitutively active, basal cAMP production may be assessed using the flashplate assay (NEN). Agonist stimulated levels of cAMP or comparison with a known constitutively active Gs coupled receptor mutant (e.g., PTH receptor T410P) may be included as positive controls.

For dopamine receptor mutants that trigger Gi/o mediated signaling, confirmation of constitutive activity may be carried out in forskolin-stimulated cells. Basal signaling in forskolin treated cells expressing the wild-type vs. constitutively active mutant are compared. The elevation in cAMP (or corresponding luciferase activity) resulting after forskolin stimulation should be decreased to a greater extent in cells expressing the constitutively active (vs. WT) receptors.

If the luciferase results suggest that constitutively active mutants are Gq coupled (i.e., activate the SRE-luciferase to a greater extent than the corresponding wild-type receptor), follow up confirmatory studies may be used to assess the basal (i.e., ligand independent) level of receptor mediated production of inositol phosphates. Agonist stimulated levels of inositol phosphate production or comparison with a known constitutively active Gq coupled receptor mutant (e.g., CCK-2R, L325E) may be included as positive controls.

In another test of constitutive activity, cells expressing constitutively active mutants may be treated with inverse agonists. Known inverse agonists for both the D1 and D2 receptors include (+)-butaclamol, haloperidol, and clozapine (Wilson et at., J. Neurochem. 77:493-504 (2001); Cai et al., Mol. Pharmacol. 56:989-996 (1999). These compounds inhibit ligand-independent signaling, and thus confirm mutation induced receptor activation.

To confirm the constitutive activity of a dopamine receptor in vivo, the function of such receptors in adult rats may also be characterized. Specifically, recombinant adeno-associated viral constructs encoding the constitutively active receptors are injected unilaterally into rat striatum and ‘circling behavior’ quantified as an index of mutant receptor efficacy. It has previously been established that asymmetric striatal dopamine receptor mediated signaling results in circling behavior, away from the side with increased receptor mediated signaling. In animal models with unilateral overexpression of wild-type D2 receptors resulting from infection with the corresponding adenoviral construct (Ikari et al., Brain Res. Mol. Brain Res. 34:315-320 (1995); Ingram et al., Exp. Gerontol. 33:793-804 (1998), peripheral administration of apomorphine (a dopamine receptor agonist) results in circling. Asymmetry in striatal dopamine 2 receptor expression has also been achieved by unilateral administration of 6-hydroxydopamine (6-OHDA), a neurotoxin that destroys nigrostriatal neurons and leads to an upregulation of D2 receptors on the 6-OHDA injected side (Sibley, Annu. Rev. Pharmacol. Toxicol. 39:313-341 (1999); Ozawa et al., J. Neural Transm. Suppl. 58:181-191 (2000); Ungerstedt et al., Brain Res. 24:485-493 (1970); Mendez et al., J. Neurosurg 42:166-173 (1975). Again, peripherally administered apomorphine results in circling behavior away from the side of increased receptor activity.

Dopamine Receptor Constructs

In a preferred approach according to the invention, a construct is generated encoding a constitutively active dopamine receptor in a vector suitable for expression in an animal. This construct is introduced into such animals through techniques well known to the skilled artisan, and described herein. In addition to the constitutively active dopamine receptor, the animal may also express a reporter system sensitive to G protein-coupled receptor activity. Examples of such reporter systems are provided herein.

In a preferred approach, a construct is generated encoding a constitutively active dopamine receptor in a viral vector. By this approach, complementary DNAs encoding each of the wild-type and mutant D1, D2L, and D2S receptors are cloned into an expression vector, for example, a rAAV transfer plasmid that directs dopamine receptor expression in neurons. In one preferred construct, the dopamine receptor is expressed from a neuron-specific enolase promoter, and the construct includes an internal ribosomal entry site driving receptor and, for animal tests, green fluorescent protein expression bicistronically (Klein et al., Brain Res. 847:314-320 (1999). Co-expression of green fluorescent protein allows rapid assessment of transduction efficiency. Similar rAAV constructs have been demonstrated to give high-level striatal expression. Any rAAV construct may be used in the methods of the invention, for example, those rAAV constructs available from the University of Florida's Gene Therapy Center (Vector Core Facility) (see, for example, http://www.gtc.ufl.edu/gtc-home.htm; http://www.gtc.ufl.edu/gtc-vraav.htm).

Recombinant AAV provides a number of advantages (Ozawa et al., J. Neural. Transm. Suppl. 58:181-191 (2000); Bjorklund et al., Brain Res. 886:82-98 (2000); Mandel et al., Experimental Neurology 159:47-64 (1999). First, the wild-type vector lacks any disease association. Second, rAAV can be used with transcripts up to 5 Kb; dopamine receptor transcripts are ˜1.5-2 Kb. Third, transgenes integrate into the host genome resulting in stable expression. Fourth, immune response to rAAV is markedly diminished since 96% of the viral genome has been removed; only genes for packaging and integration remain intact. Fifth, rAAV can transduce both non-dividing and dividing cells. Sixth, well-documented, high efficiency transduction occurs in striatal neurons. And, seventh, high-level expression is achieved for at least 2-6 months post infection.

For each dopamine receptor, virus encoding wild-type and a constitutively active mutant (ideally with 50-100% activity, relative to the dopamine induced maximum, as assessed by in vitro assays) are generated. An empty rAAV vector is utilized as an additional negative control.

As each preparation of rAAV is completed, constructs are tested in HEK293 cells to ensure adequate receptor expression as well as to confirm basal receptor mediated signaling. After rAAV infection, receptor densities are determined using homologous competition binding experiments with tritiated SCH 23390 or tritiated spiperone, selective radioligands for the D1 or D2 receptor, respectively Ozawa et al., J. Neural. Transm. Suppl. 58:181-191 (2000); Ingram et al., Mech. Ageing Dev. 116:77-93 (2000). Constitutive activity is verified with the appropriate luciferase reporter assay, SMS-luciferase for the D1 receptor and SRE-luciferase/Gq5i for the D2 receptor. Alternatively, constitutive activity of the D1 receptor may be assayed directly by measurement of cAMP levels.

Constitutively Active Dopamine Receptor Animals

Animals expressing a constitutively active dopamine receptor can be used in drug screening or for testing therapeutic efficacy of receptors or compounds, for example, prior to human clinical trials. Methods for drug screening are well known in the art, and are described herein. In one example, animals expressing a constitutively active dopamine receptor receive a test compound. The effect of the test compound on G protein-coupled receptor activity is then assayed (for example, by reporter output), using standard methods well known in the art; examples of such assays are described herein. The effect of the test compound on the animal is assessed relative to a control group of animals that did not receive the test compound.

Alternatively, assays are carried out that measure phenotypic output. In one particularly preferred approach, constructs that include rAAV encoding a constitutively active mutant receptor, a wild-type receptor, or no receptor are tested in rodents (for example, male Sprague-Dawley rats (250-300 g) of comparable age) for effects on circling behavior. Ten animals comprise each group. In these tests, each rat receives a single unilateral injection of rAAV, 4 μl of a ˜10¹² particles per ml stock, into the dorsolateral striatum (DLS). This dose of virus is similar to ones used in earlier studies that successfully targeted the striatum (Ozawa et al., J. Neural. Transm. Suppl. 58:181-191 (2000); Bjorklund et al., Brain Res. 886:82-98 (2000); Klein et al., Brain Res. 847:314-320 (1999). A rAAV construct encoding GFP may be used to confirm that the striatal coordinates for injection (as per the Paxinos and Watson, Stereotaxic Atlas of the Rat Brain, 1998) target the DLS. In these animals it may also be determined whether and to what extent there is expression of GFP outside the targeted region; appropriate adjustments in dose, number of injections, and/or coordinates may be made based on these measurements.

Circling behavior in ten adult male rats is compared with equal numbers of controls. Animals are evaluated every other day for the onset of circling behavior by placing rats in a circular chamber (diameter=36 cm.) and monitoring behavior. Circling is recorded and quantified using the Ethovision video monitoring system (Noldus Information Technologies, Sterling, Va.). If no spontaneous circling behavior is evident after 5 weeks, animals are evaluated after peripheral administration of apomorphine, a dopamine receptor agonist. The 5-week interval allows ample time to achieve a stable level of receptor expression levels (Ozawa et al., J. Neural. Transm. Suppl. 58:181-191 (2000); Bjorklund et al., Brain Res. 886:82-98 (2000). Apomorphine-induced circling away from the side of the rAAV injection indicates that the viral construct induced receptor overexpression/asymmetry. At the same time, a lack of spontaneous circling in the absence of drug treatment suggests that the level of receptor expression and/or basal activity was not sufficient to induce spontaneous circling. In this case, expression levels may be increased by utilizing a higher dose of the injected rAAV construct and/or by widening the striatal field injected (Ozawa et al., J. Neural. Transm. Suppl. 58:181-191 (2000); Bjorklund et al., Brain Res. 886:82-98 (2000). As detailed below, the level of receptor expression is quantified by receptor autoradiography to monitor how alterations in dose-injection pattern influence striatal receptor density. Alternatively, the rAAV constructs may be further optimized by identifying additional point mutations that confer a greater degree of constitutive activity, as described above.

Once results are known with each construct individually, a combination of the constitutively active D2L and D1 rAAV constructs may be injected in parallel in equal amounts. A combination of corresponding wild-type constructs are used as a control.

In addition to enhancing locomotor behavior, excess receptor activity might result in abnormal movements including writhing and/or tremors. In this case, a lower dose of the injected rAAV construct(s) is used and/or the striatal field injected is narrowed. Alternatively, the relevant rAAV construct(s) may be made using a less constitutively active receptor mutant.

Receptor expression is assessed in all rats (i.e., those that circle as well as those that do not) after completion of circling behavior studies. Rats are anesthetized with pentobarbital. The animals are then perfused transcardially with phosphate buffered saline followed by 4% paraformaldehyde w/sucrose. Brains are removed, frozen, and cut into transverse sections (20 microns) that extend through the striatum bilaterally. Since the rAAV constructs used in the animal tests encode green fluorescent protein (GFP) in parallel with the receptors, GFP expression provides a rapid index of protein expression. The brain sections also allow assessment of (i) tissue damage, (ii) accuracy of cannula placement, and (iii) dorsolateral striatum specific expression. To quantify striatal receptor expression, frozen brain sections are assessed using receptor autoradiography with subtype selective radioligands, tritiated spiperone for D2 receptors and tritiated SCH 23390 for D1 receptors (Sibley, D. R., Annu. Rev. Pharmacol. Toxicol. 39:313-341 (1999); Xu et al., Cell 79:729-742 (1994); Ingram et al., Mech. Ageing Dev. 116:77-93 (2000). The autoradiographic signals are measured using the Alpha Innotech Corp. ChemiImager 4400 densitometer. Parallel controls include animals injected with an empty rAAV as well as with rAAV encoding wild-type receptors.

Animals of the instant invention may also be used to assay receptors or compounds useful for Parkinson's disease. For example, animals may be treated with compounds to further induce Parkinson's disease symptoms prior to use in the assays described herein. Such treatments are well known to the skilled artisan. In one particular example, 6 hydroxydopamine (6-OHDA) has been used to generate a rat model of Parkinson's disease published by Diaz et al. (Rodriguez Diaz et al., Behav. Brain Res. 122:79-92 (2001); Breese, G. R., et al., Br. J. Pharmacol. 42:88-99 (1971); Rodriguez et al., Exp. Neurol. 169:163-181 (2001). In this model, 6-OHDA produces Parkinsonian-like symptoms, including a decrease in spontaneous locomotor activity and an accompanying increase in chewing behavior and catalepsy. Animals expressing constitutively active dopamine receptors and treated with 6-OHDA provide a sensitive system in which to assay the potential therapeutic effect of constitutive dopamine receptor activity or to assay for dopamine receptor agonists. Test compounds that increase spontaneous locomoter activity or, for example, decrease chewing behavior and catalepsy in constitutively active dopamine receptor expressing animals or in 60HDA-treated animals of the instant invention are useful for the treatment of human Parkinson's disease.

In another example, as discussed above, it has previously been established that asymmetric striatal dopamine receptor-mediated signaling results in circling behavior, away from the side with increased receptor-mediated signaling. A test compound may be administered directly into the brains of an animal of the instant invention. The effect of asymmetric administration of a test compound may then be assessed by documenting circling. Receptor expression or test compounds that induce circling behavior are identified as receptor or compounds that increase signaling. Such receptors or compounds may be useful for the treatment of Parkinson's disease. In addition, such receptors or compounds may also be useful for memory enhancement as well as for improving cardiovascular or renal function.

### EXAMPLE 3

### Constitutively Active Melanocortin-4 Receptor

This example describes the identification of constitutively active melanocortin-4 (MC-4) receptors and the use of nucleic acids encoding these receptors to generate animals useful in drug-screening and/or testing of therapeutic efficacy of receptors or compounds.

Identifying Regions of Homology and Generating MC-4 Receptor Mutants

As shown in FIG. 8, the “DRY” motif is conserved between the Class A G protein-coupled, oxytocin, vasopressin-V-2, cholecystokinin-A (CCK-A), MC-4, and α1B adrenergic receptors (FIG. 8). Based on this homology, plus precedent that substitution of aspartic acid within the DRY motif results in constitutively active oxytocin, vasopressin V-2, CCK-A, and α1B receptors, we hypothesized that substitution of the D (Asp) residue at position 146 of MC-4 by a non-charged residue would yield a constitutively active receptor (the MC-4 sequence is available as Genebank Accession is L08603). An Asp146Met mutant MC-4 receptor was generated using routine methods.

Assaying of Mutant MC-4 Receptors for Constitutive Activity

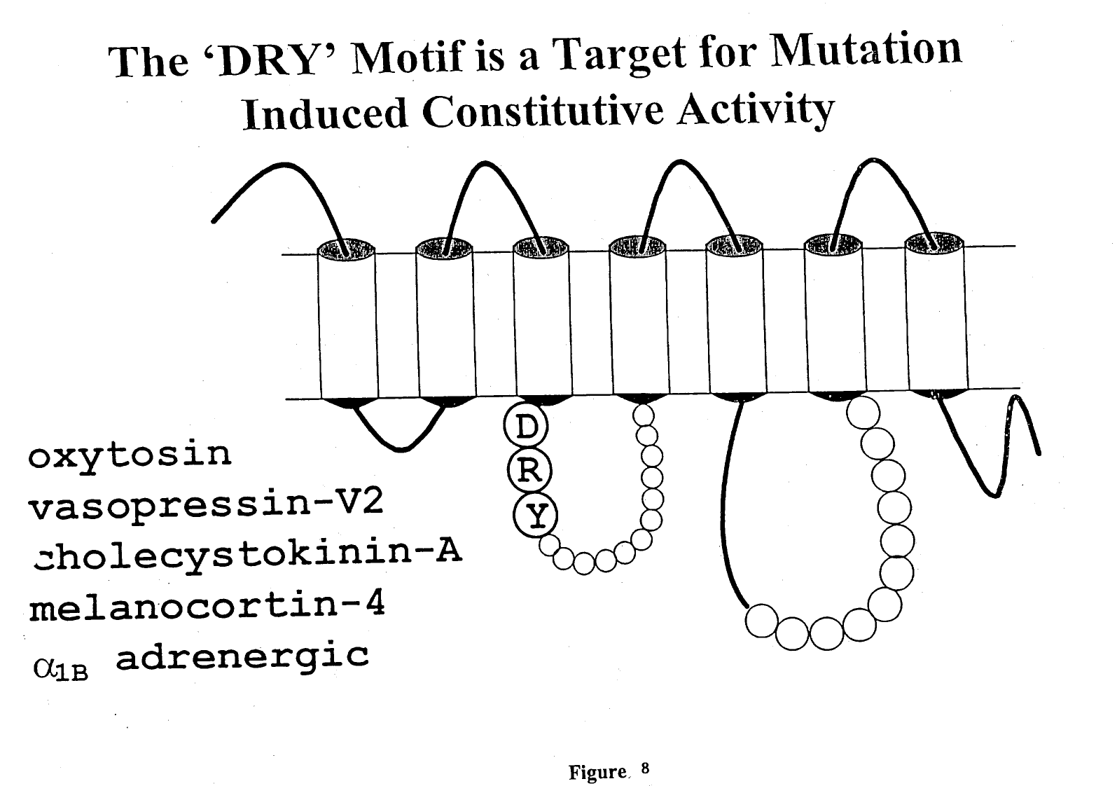

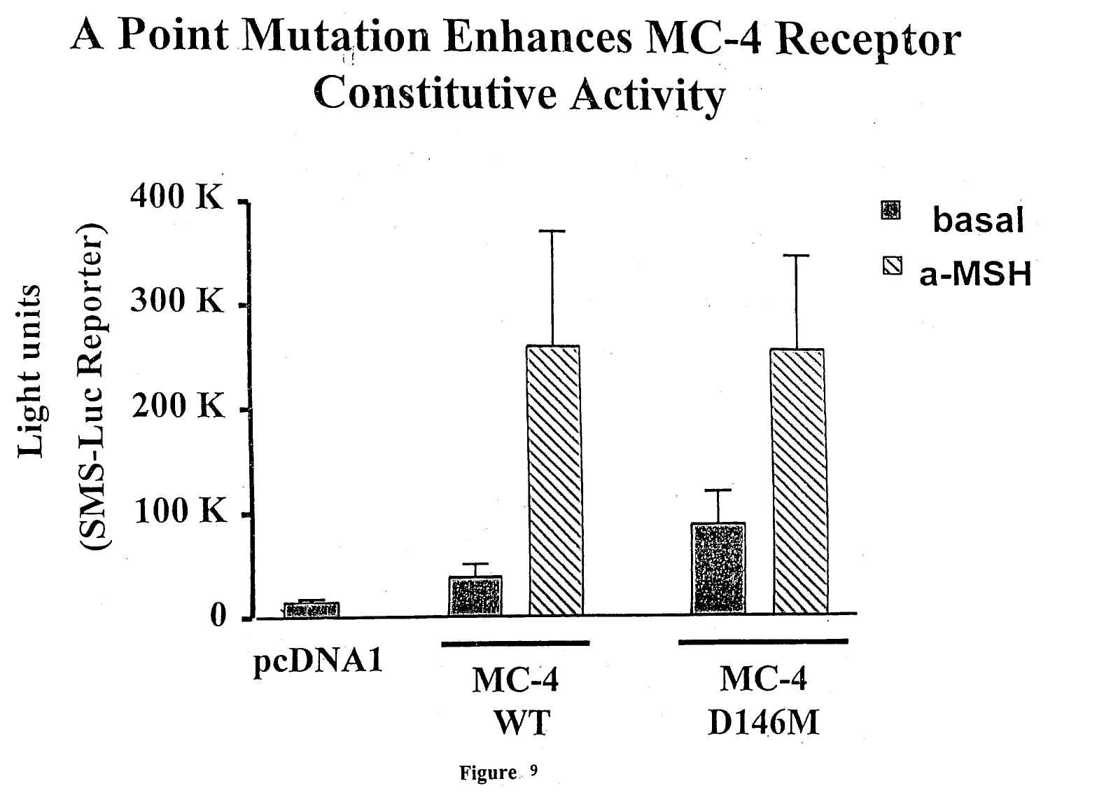

As demonstrated in FIG. 9, the reporter system assay was capable of detecting constitutive activity of the mutant Asp146Met MC-4 receptor. Briefly, HEK293 cells were cotransfected, as described above, with an expression vector encoding either the wild-type MC-4 receptor or the Asp146Met mutant MC-4 receptor and the reporter construct, SMS-Luc. As a negative control, cells were transfected with SMS-Luc and pcDNA1. Basal and ligand (αMHS) induced activity of the negative control, the wild-type MC-4 receptor, and the Asp146Met mutant MC-4 receptor were measured using the luciferase assay described above. The Asp146Met mutant MC-4 receptor mutant clearly exhibited a higher basal level activity than its wild-type counterpart. This mutant also exhibited constitutive activity in a cAMP assay (FIG. 2). Other constitutively active MC-4 receptors may be generated by this or any other approach and introduced as transgenics into animals of the invention

Constitutively Active MC-4 Receptor Animals

In a preferred approach according to the invention, a construct is generated encoding a constitutively active MC-4 receptor in a vector suitable for expression in an animal. Preferably, the constitutively active MC-4 receptor is expressed in the brain (as described above) and most preferably in the neurons of the hypothalamus (Harrold et al., Diabetes 48:267 (1999); Broberger et al., Physiol. Behav. 74:669 (2001)). Such expression vectors are well known in the art. This construct is used to generate animals through techniques well known to the skilled artisan, and described herein. In addition to the constitutively active MC-4 receptor, the animal may also express a reporter system sensitive to G protein-coupled receptor activity. Examples of such reporter systems are provided herein.

Drug screens for test compounds that modulate the MC-4 receptor may be carried out in animals expressing constitutively active MC-4 receptors. These techniques may also be used to test therapeutic efficacy of receptors or compounds proteins, for example, prior to or in conjuction with human clinical trials. The effect of the receptor or test compound on MC-4 receptor activity may be assayed using any standard method known in the art. The effect of the constitutively active receptor or test compound on the animal is assessed relative to a control group of animals that did not receive the constitutively active receptor or test compound.

The MC-4 receptor is a G protein-coupled seven transmembrane receptor expressed in the brain that has been implicated in a maturity onset obesity syndrome associated with hyperphagia, hyperinsulinemia, and hyperglycemia in mice (Huszar et al. Cell 88:131-41). Specifically, chronic antagonism of the MC-4 receptor by the agouti polypeptide induces a novel signaling pathway that increases glucose tolerance and results in increased body weight. Assays for glucose tolerance are well known to the skilled artisan. Accordingly, any such assay (for example, measurement of body weight or food intake) may be used as a phenotypic output for MC-4 receptor activity.

Test compounds or constitutively active receptors that modulate MC-4 receptor activity can be used to control body weight or to treat obesity. Such compounds may be identified using animals of the invention to assay for the modulation of G protein-coupled receptor activity. For example, a reporter construct may be used to detect changes in receptor activity. Alternatively, such compounds may be identified by detecting a change in the body weight or food intake of an animal treated with a test compound, relative to a control animal not receiving the test compound.

Compounds that modulate MC-4 activity may also be useful in the treatment of hyperinsulinemia and/or hyperglycemia. Such compounds may be identified using reporter constructs that allow the detection of a change in G protein-coupled receptor activity. Alternatively, animals of the invention may be assayed for glucose tolerance, food intake, or assessment of weight gain. Such assays are standard in the art (see, for example, Kopin et al., J. Clin. Invest. 103:383 (1999)).

### EXAMPLE 4

### Constitutively Active β2 Adrenergic Receptors

This example describes the identification of hypersensitive β2 adrenergic receptors and the use of nucleic acids encoding these receptors to generate animals useful in drug-screening and/or the testing of therapeutic efficacy of receptors or compounds, for example, in conjunction with clinical trials.

Identifying Regions of Homology and Generating Constitutively Active β2 Adrenergic Receptor

As described in Samama et al. (J. Biol. Chem. 268(7):4625-4636, 1993), a constitutively active mutant of the β2 adrenergic receptor was generated by replacing the C-terminal portion of the third intracellular loop of the β2 adrenergic receptor with the homologous region of the 1B adrenergic receptor. This conservative substitution led to agonist independent activation of the β2 adrenergic receptor. In addition, the constitutively active receptor has an increased intrinsic affinity for β2 adrenergic receptor agonists and partial agonists, as well as an increased potency, and is therefore also hypersensitive. Other constitutively active β2 adrenergic receptors may be generated by this technique or any other method described herein or known in the art.

Constitutively Active β62 Adrenergic Receptor Expressing Animals

Agonists to the β2 adrenergic receptor have been widely used to treat asthma. In fact, inhaled beta-adrenergic agonists are the most commonly used treatments for asthma today (Drazen et al., Am. J. Respir. Care Critical Med. 162(1):75-80 (2000)). In addition, polymorphisms in the gene encoding the β2 adrenergic receptor have been identified and correlated with asthma severity (Holloway et al., Clin. Exp. Allergy 30(8):1097-103 (2000)). Thus, according to the present invention, constitutively active β2 adrenergic receptors expressed in animals are useful for the identification of receptors or therapeutic compounds for the treatment and prevention of asthma.

Compounds that modulate β2 adrenergic receptor activity may be identified using animals of the invention by detecting a change in G protein-coupled receptor activity. These constitutively active β2 adrenergic receptors are expressed in the airways (see, for example, Skoner, J. Allergy Clin. Immunol. 106:5158 (2000)). Changes in activity may be assayed, for example, using a reporter system to measure changes in receptor signaling. Alternatively, useful therapeutic receptors or compounds may be identified by detecting a change in the phenotype of the animal relative to an animal that did not receive the compound. The effects of candidate compounds are preferably assayed by comparing animals in pulmonary function tests, or by airway hyperresponsiveness (see, for example, DeSanctis et al., J. Allergy Clin. Immunol. 108:11 (2001)).

### EXAMPLE 5

### Constitutively Active α1 Adrenergic Receptors

This example describes the identification of constitutively active α1 adrenergic receptors and the use of nucleic acids encoding these receptors to generate animals useful in drug-screening and for the testing of therapeutic efficacy of receptors or compounds.

Identification of Constitutively Active α1 Adrenergic Receptors

As illustrated in FIG. 1, numerous exemplary α1 adrenergic receptors have been identified that have constitutive activity. Indeed, nineteen different amino acid substitutions of the Ala at position 293 of the α1 adrenergic receptor result in constitutive activity of the receptor (Kjelsberg et al., J. Biol. Chem. 267(3):1430-1433 (1992)). Additional constitutively active mutants of the α1 adrenergic receptor include mutants of the DRY motif at the junction between transmembrane domain III and intracellular loop 2. These mutants include the Asp142Ala mutant (Scheer et al., Mol. Pharm. 57(2):219-231 (2000)) and the Arg143Lys mutant (Scheer et al., Proc. Natl. Acad. Sci USA 94(3):808-813 (1997)). Another constitutively active mutant of the α1 adrenergic receptor is the Asn63Ala mutant (Scheer et al., supra (1997)). Mutation of this conserved Asn63 residue located N-terminal to the DRY motif frequently leads to constitutive activity in a variety of other G-protein-coupled receptors (see FIG. 7). Other constitutively active α1 adrenergic receptors include the Cys128Phe mutant (in transmembrane domain III) (Perez et al., Mol. Pharmacol. 49(1):112-122 (1996)); the Ala293Glu mutant (carboxyl end of IC3) (Perez et al., supra); and the Ala204Val mutant (transmembrane domain V) (Hwa et al., Biochemistry 36(3):633-639 (1997). Other mutants include those described in Allen et al. (Proc. Natl. Acad. Sci. USA 88(24):11354-11358 (1991) and shown in FIG. 1, page 2).

Constitutively Active α1 Adrenergic Receptor Animals

Phenylepinepherine is a commonly used agonist of the α1 adrenergic receptor for the treatment of nasal congestion. Thus, according to the present invention, constitutively active α1 adrenergic receptors are useful in the identification of treatments for nasal congestion. Candidate compounds can be administered to animals expressing a constitutively active α1 adrenergic receptor nucleic acid (e.g., to the surfaces of nasal passages, e.g., via a nasal spray), and the effects of these candidate compounds on G protein-coupled receptor activity may be detected, for example, using a reporter system. Examples of such reporter systems are provided herein. Alternatively, the effect of a candidate compound on G protein-coupled receptor activity may be assayed in an animal expressing a constitutively active α1 adrenergic receptor in a phenotypic screen, for example, a screen for nasal congestion (see, for example, Koss et al., Am. J. Rhinol. 16:49 (2002)).

### EXAMPLE 6

### Constitutively Active Glucagon-Like Peptide-1 Receptor

This example describes the use of nucleic acids encoding constitutively active glucagon-like peptide-1 (Glp-1) receptors to generate animals useful in drug screening and/or for testing therapeutic efficacy of constitutively active receptors or candidate compounds.

The (GLP-1) receptor is a G protein-coupled receptor (Graziano et al. (Biochem. Biophys. Res. Commun. 196(1):141-146 (1993)). The human and rat GLP-1 receptor genes have been cloned and compared and regions of conservation identified (Dillon et al., Endocrinology 133(4):1907-1910, (1993)). GLP-1 receptor is activated by GLP-1, a hormone secreted from the distal gut that stimulates basal and glucose-induced insulin secretion and proinsulin gene expression (Dillon et al., supra). GLP-1 is associated with involvement of the CNS in the inhibition of upper gastrointestinal motility (van Dijk et al., Neuropeptides 33(5):406-414 (1999)).

Constitutively active GLP-1 receptors may be generated and used to produce animals, for example, by the methods described herein. The constructs preferably provide for GLP-1 expression in pancreatic β-cells or in the brain, most preferably, in the hypothalamus) (see above). These animals are then used to identify therapeutic compounds or to test compounds for their therapeutic efficacy for the treatment of diabetes. Such therapeutic compounds may be identified using animals of the invention to assay for the modulation of GLP-1 receptor activity. For example, a reporter construct may be used to detect changes in receptor activity. Alternatively, such compounds may be tested in a behavioral or drug response assay; such assays include glucose tolerance tests or assays for food intake.

### EXAMPLE 7

### Constitutively Active Cholecystokinin-B/Gastrin Receptors (CCK-BR)

This example describes the identification of constitutively active CCK-BR receptors (Beinbom et al., J. Biol. Chem. 273(23):14146-14151, 1998 and Beinbom et al., Gastroenterology 110(suppl.):A1059, 1996), and the use of nucleic acids encoding these receptors to generate animals useful in drug-screening and/or to test receptors or compounds for therapeutic efficacy.

Identifying Regions of Homology and Generating Mutant CCK-BR Receptors

Molecular characterization of the third intracellular loop of the human CCK-BR led to the identification of a point mutation (Leu325Glu) which results in constitutive CCK-BR activity (see, Beinborn et al. supra (1996)). Briefly, the strategy was based on the theory that domain swapping between related polypeptides with different second messenger couplings could yield receptors having increased basal activity. Segments of 4-5 amino acids were substituted in the third intracellular loop of the CCK-BR with corresponding sequences from the vasopressin 2 receptor, a protein with 30% amino acid identity to CCK-BR. However, these proteins are coupled to different signal transduction pathways. CCK-BR is coupled to phospholipase C activation, whereas the vasopressin 2 receptor is coupled to adenylyl cyclase as the predominant signal transduction pathway (Beinborn et al., supra (1996)).

Assaying Mutant CCK-BR Receptors for Constitutive Activity

As described in Beinborn et al., recombinant receptors were transiently expressed in COS-7 cells and ligand affinities were assessed by ¹²⁵I CCK-8 competition binding experiments. In addition, phospholipase C-mediated production of inositol phosphate was measured in the absence and in the presence of agonists. One of the block substitutions from the vasopressin 2 receptor, 250AHVSA, conferred agonist-independent constitutive activity when introduced into the corresponding region of the third intracellular loop of the CCK-BR. The mutant CCK-BR triggered a 10-fold higher basal turnover of inositol phosphate compared to wild-type CCK-BR. Substitution of 253SA and even 253S alone within the same segment was sufficient to confer constitutive activity as well (Beinborn et al., (Abstract) supra (1996).)

Additional studies were carried out as described in Beinbom et al. (supra (1998)). In particular, the Leu325Glu CCK-BR mutant triggers constitutive production of inositol phosphates to levels exceeding wild-type CCK-BR (Beinborn et al., FIG. 1A supra (1998)). Briefly, the human wild-type CCK-BR and the constitutively active Leu325Glu CCK-BR mutant were transiently expressed in COS-7 cells. Control cells (“no receptor”) were transfected with the empty expression vector, pcDNA1. Cells were pre-labeled overnight with myo-[³H]inositol and then stimulated with ligand for 30 to 60 minutes in the presence of 10 mM LiCl. The constitutively active CCK-BR mutant is clearly distinguished from the wild-type receptor by its ability to trigger inositol phosphate production in the absence of agonist.

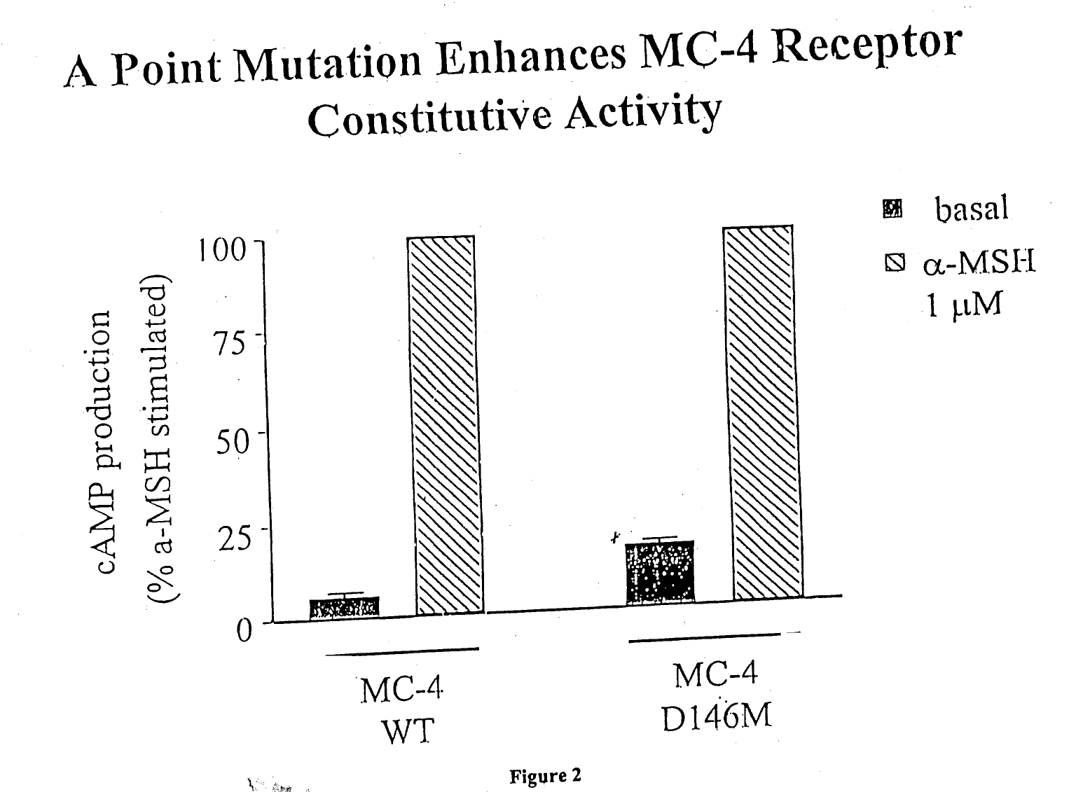

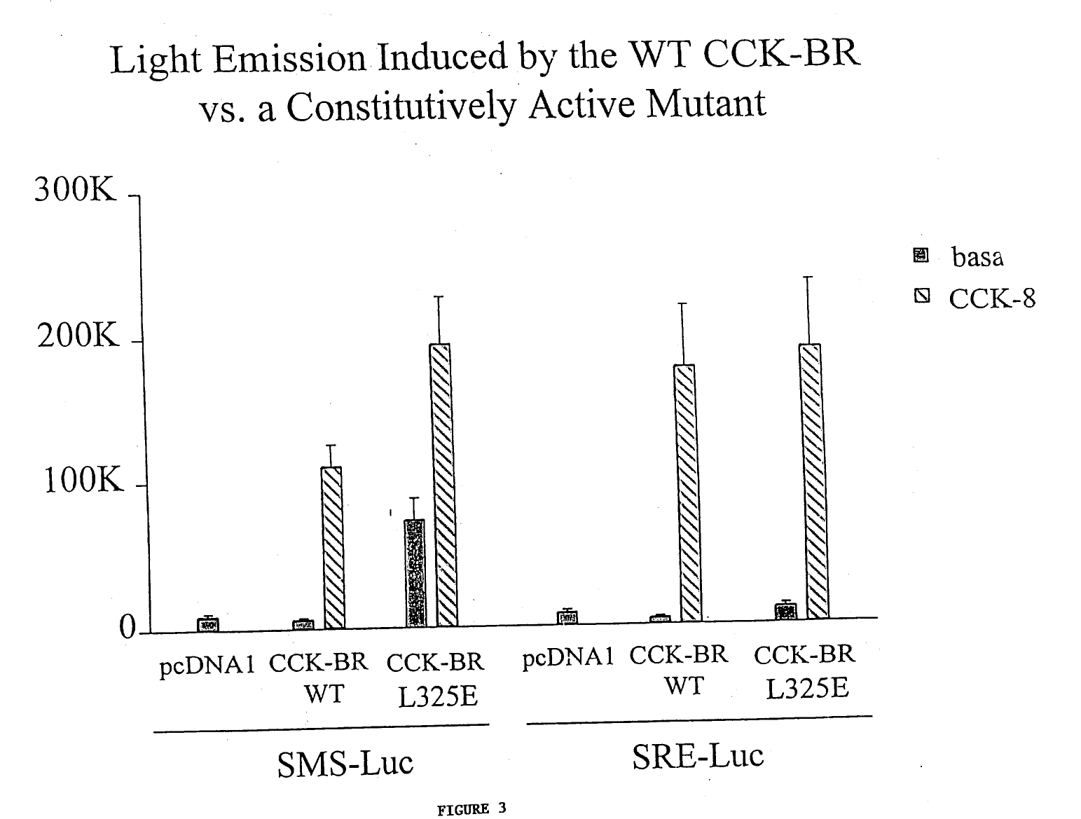

In addition to these studies, luciferase assays were performed to measure the constitutive activity of the Leu325Glu CCK-BR mutant. HEK293 cells were transfected (as described above) with SMS-Luc and an expression vector encoding any one of pcDNA1, wild-type CCK-BR, or Leu325Glu CCK-BR. As demonstrated in the left panel of FIG. 3, the Leu325Glu CCK-BR mutant has increased basal level activity compared to the wild-type CCK-BR.

Any other constitutively active CCK-BR may also be used in the invention.

Constitutively Active CCK-BR Animals

CCK-BR is a G protein-coupled receptor that has been implicated in modulating memory, anxiety, and pain perception, as well as in regulating gastrointestinal mucosal growth and secretion (Beinborn et al. supra, 1998). Thus an animal expressing a constitutively active CCK-BR may be used to identify therapeutic receptors or compounds or to test therapeutic efficacy for the treatment of a wide range of diseases, including diseases that produce memory deficits. Such animals are generated by introduction into the animal of an expression construct that produces the constitutively active CCK-BR in the stomach. Candidate compounds that modulate G protein-coupled receptors may be identified using animals of the invention to assay for the modulation of G protein-coupled receptor activity. For example, a reporter construct may be used to detect changes in receptor activity. Alternatively, such compounds may be tested in behavioral or drug response assays, for example, by detecting a change in memory or assaying for stomach ulcers. Useful receptors or therapeutic compounds act as antagonists of the CCK-BR.

Other Embodiments

From the foregoing description, it will be apparent that variations and modifications may be made to the invention described herein to adopt it to various usages and conditions. Such embodiments are also within the scope of the following claims.

All patents and publications mentioned in this specification are herein incorporated by reference to the same extent as if each independent publication was specifically and individually indicated to be incorporated by reference.

While the invention has been described in connection with specific embodiments thereof, it will be understood that it is capable of further modifications. This application is intended to cover any variations, uses, or adaptations following, in general, the principles of the invention and including such departures from the present disclosure within known or customary practice within the art to which the invention pertains and may be applied to the essential features hereinbefore set forth.

What is claimed is:

1. A method of determining whether a constitutively active G protein-coupled receptor has potential therapeutic activity, said method comprising:
(a) introducing a nucleic acid encoding a constitutively active G protein-coupled receptor into a non-human animal under conditions which allow expression of said constitutively active G protein-coupled receptor in said animal in a tissue which normally expresses said receptor; and
(b) without breeding said animal, assaying a phenotypic output of said expression of said constitutively active G protein-coupled receptor, whereby a positive phenotypic output relative to a control animal lacking expression of said constitutively active G protein-coupled receptor indicates that said constitutively active G protein-coupled receptor has potential therapeutic activity.

2. A method of determining whether a G protein-coupled receptor is a candidate drug screening target, said method comprising:
(a) introducing a nucleic acid encoding a constitutively active G protein-coupled receptor into a non-human animal under conditions which allow expression of said constitutively active G protein-coupled receptor in said animal in a tissue which normally expresses said receptor; and
(b) without breeding said animal, assaying a phenotypic output of said expression of said constitutively active G protein-coupled receptor, whereby either a positive phenotypic output or a negative phenotypic output relative to a control animal lacking expression of said constitutively active G protein-coupled receptor indicates that said G protein-coupled receptor or a constitutively active variant thereof is a candidate drug screening target.

3. A method of identifying a candidate therapeutic compound, said method comprising:
(a) introducing a nucleic acid encoding a constitutively active G protein-coupled receptor into a non-human animal under conditions which allow expression of said constitutively active G protein-coupled receptor in said animal in a tissue which normally expresses said receptor;
(b) without breeding said animal, assaying a phenotypic output of said expression of said constitutively active G protein-coupled receptor, whereby either a positive phenotypic output or a negative phenotypic output relative to a control animal lacking expression of said constitutively active G protein-coupled receptor indicates that said G protein-coupled receptor or a constitutively active variant thereof is a drug screening target for a therapeutic compound;
(c) contacting said G protein-coupled receptor or constitutively active variant thereof identified in step (b) with a candidate compound; and
(d) measuring the activity of said G protein-coupled receptor or constitutively active variant thereof in the presence and in the absence of said candidate compound, whereby a candidate therapeutic compound is identified as a compound that alters the activity of said G protein-coupled receptor or constitutively active variant thereof.

4. The method of claim 2 or 3, wherein expression of said constitutively active G protein-coupled receptor results in a positive phenotypic output and said G protein-coupled receptor or said constitutively active variant thereof is used as a candidate drug screening target for an agonist.

5. The method of claim 2 or 3, wherein expression of said constitutively active G protein-coupled receptor results in a negative phenotypic output and said G protein-coupled receptor or said constitutively active variant thereof is used as a candidate drug screening target for an inverse agonist or antagonist.

6. The method of any of claims 1-3, wherein said G protein-coupled receptor has a peptide, lipid, small molecule, amino acid, or biogenic amine ligand.

7. The method of claim 4, wherein said agonist is selected from the group consisting of a peptide, lipid, small molecule, amino acid, and biogenic amine.

8. The method of claim 5, wherein said inverse agonist or antagonist is selected from the group consisting of a peptide, lipid, small molecule, amino acid, and biogenic amine.

9. The method of any of claims 1-3, wherein said G protein-coupled receptor is an orphan receptor.

10. The method of any of claims 1-3, wherein said G protein-coupled receptor is a human receptor.

11. The method of any of claims 1-3, wherein said constitutively active G protein-coupled receptor is a dopamine receptor.

12. The method of claim 11, wherein said nucleic acid expressing said constitutively active dopamine receptor is expressed in neurons and encodes a constitutively active D1 receptor.

13. The method of claim 11, wherein said nucleic acid expressing said constitutively active dopamine receptor is expressed in neurons and encodes a constitutively active D2 receptor.

14. The method of claim 11, wherein said nucleic acid expressing said constitutively active dopamine receptor is expressed in neurons and encodes a constitutively active D2L receptor.

15. The method of claim 11, wherein said nucleic acid encoding a constitutively active dopamine receptor is expressed in neurons and encodes a constitutively active D2S.

16. The method of any of claims 1-3, wherein said constitutively active G protein-coupled receptor is a mu opioid receptor.

17. The method of claim 16, wherein said nucleic acid expressing said constitutively active mu opioid receptor is expressed in neurons.

18. The method of claim 16, wherein said constitutively active mu opioid receptor comprises an Asparagine at amino acid 150.

19. The method of any of claims 1-3, wherein said constitutively active G protein-coupled receptor is a melanocortin-4 receptor.

20. The method of any of claims 1-3, wherein said constitutively active G protein-coupled receptor is a β2 adrenergic receptor.

21. The method of any of claims 1-3, wherein said constitutively active G protein-coupled receptor is an α1 adrenergic receptor.

22. The method of any of claims 1-3, wherein said constitutively active G protein-coupled receptor is a cholecystokinin-B/gastrin (CCK-BR) receptor.

23. The method of any of claims 1-3, wherein said constitutively active G protein-coupled receptor is a glucagon-like peptide (GLP-1) receptor.

24. The method of claim 23, wherein said nucleic acid expressing said constitutively active GLP-1 receptor is expressed in neurons.

25. The method of any of claims 1-3, wherein said animal is a vertebrate.

26. The method of claim 25, wherein said vertebrate is a rodent.

27. The method of claim 26, wherein said rodent is a mouse or rat.

28. The method of any of claims 1-3, wherein said nucleic acid encoding said constitutively active G protein-coupled receptor is introduced into said animal using a viral vector.

29. The method of claim 28, wherein said viral vector is an AAV vector.

30. The method of any of claims 1-3, wherein said constitutively active G protein-coupled receptor is overexpressed in said tissue of said animal.


SEQUENCE LISTING (87 sequences)

SEQ ID NO: 1 - protein, 398 aa, Rattus norvegicus
MDSSTGPGNTSDCSDPLAQASCSPAPGSWLNLSHVDGNQSDPCGLNRTGLGGNDSLCPQTGSPSMVTAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPILIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSTRVRQNTREHPSTANTVDRTNHQLENLEAETAPLP

SEQ ID NO: 2 - protein, 11 aa, Artificial Sequence
  Synthetic fragment
VSIVLETTIIL

SEQ ID NO: 3 - protein, 11 aa, Artificial Sequence
  Synthetic fragment
RERKATKTLGI

SEQ ID NO: 4 - protein, 11 aa, Artificial Sequence
  Synthetic fragment
NEQKACKVLGI

SEQ ID NO: 5 - protein, 11 aa, Artificial Sequence
  Synthetic fragment
NEDDASKVLGI

SEQ ID NO: 6 - protein, 11 aa, Artificial Sequence
  Synthetic fragment
FAIVGNILVIL

SEQ ID NO: 7 - protein, 11 aa, Artificial Sequence
  Synthetic fragment
CAISIDRYIGV

SEQ ID NO: 8 - protein, 11 aa, Artificial Sequence
  Synthetic fragment
CAISIDRYIGV

SEQ ID NO: 9 - protein, 11 aa, Artificial Sequence
  Synthetic fragment
AVDVLCCTASI

SEQ ID NO: 10 - protein, 11 aa, Artificial Sequence
  Synthetic fragment
REKKAAKTLGI

SEQ ID NO: 11 - protein, 12 aa, Artificial Sequence
  Synthetic fragment
EEPFYALFSSLG

SEQ ID NO: 12 - protein, 9 aa, Artificial Sequence
  Synthetic fragment
SREKKAAKT

SEQ ID NO: 13 - protein, 14 aa, Artificial Sequence
  Synthetic fragment
KFSREKKAAKTLGI

SEQ ID NO: 14 - protein, 10 aa, Artificial Sequence
  Synthetic fragment
EKRFTFVLAV

SEQ ID NO: 15 - protein, 13 aa, Artificial Sequence
  Synthetic fragment
SLVKEKKAARTLS

SEQ ID NO: 16 - protein, 10 aa, Artificial Sequence
  Synthetic fragment
KKVTRTILAA

SEQ ID NO: 17 - protein, 13 aa, Artificial Sequence
  Synthetic fragment
TWTPYNIMVLVNT

SEQ ID NO: 18 - protein, 21 aa, Artificial Sequence
  Synthetic fragment
AILLAFIITWTPYNIMVLVST

SEQ ID NO: 19 - protein, 15 aa, Artificial Sequence
  Synthetic fragment
YNIMVLVSTFCDKCV

SEQ ID NO: 20 - protein, 11 aa, Artificial Sequence
  Synthetic fragment
RKAFQGLLCCA

SEQ ID NO: 21 - protein, 14 aa, Artificial Sequence
  Synthetic fragment
FCLKEHKALKTLGI

SEQ ID NO: 22 - protein, 15 aa, Artificial Sequence
  Synthetic fragment
SFKMSFKRETKVLKT

SEQ ID NO: 23 - protein, 15 aa, Artificial Sequence
  Synthetic fragment
APDTSIKKETKVLKT

SEQ ID NO: 24 - protein, 11 aa, Artificial Sequence
  Synthetic fragment
FVCCWLPFFIL

SEQ ID NO: 25 - protein, 11 aa, Artificial Sequence
  Synthetic fragment
FMISLDRYCAV

SEQ ID NO: 26 - protein, 12 aa, Artificial Sequence
  Synthetic fragment
FMVLGGFTSTLY

SEQ ID NO: 27 - protein, 10 aa, Artificial Sequence
  Synthetic fragment
GCNLEGFFAT

SEQ ID NO: 28 - protein, 14 aa, Artificial Sequence
  Synthetic fragment
MTIPAFFAKSAAIY

SEQ ID NO: 29 - protein, 11 aa, Artificial Sequence
  Synthetic fragment
VVLAIERYVVV

SEQ ID NO: 30 - protein, 11 aa, Artificial Sequence
  Synthetic fragment
RMVIIMVIAFL

SEQ ID NO: 31 - protein, 11 aa, Artificial Sequence
  Synthetic fragment
PAFFAKSAAIY

SEQ ID NO: 32 - protein, 11 aa, Artificial Sequence
  Synthetic fragment
VVLAIERYVVV

SEQ ID NO: 33 - protein, 10 aa, Artificial Sequence
  Synthetic fragment
FRKLCNCKQK

SEQ ID NO: 34 - protein, 11 aa, Artificial Sequence
  Synthetic fragment
AIISMNLYSSI

SEQ ID NO: 35 - protein, 12 aa, Artificial Sequence
  Synthetic fragment
LLFIICWLPFQI

SEQ ID NO: 36 - protein, 11 aa, Artificial Sequence
  Synthetic fragment
ASVSFNLYASV

SEQ ID NO: 37 - protein, 11 aa, Artificial Sequence
  Synthetic fragment
LFYGFLGKKFK

SEQ ID NO: 38 - protein, 25 aa, Artificial Sequence
  Synthetic fragment
LVIWVAGFRMTHTVTTISYLNKAVA

SEQ ID NO: 39 - protein, 25 aa, Artificial Sequence
  Synthetic fragment
LVVWVTAFEAKRTINAIWFLNLAVA

SEQ ID NO: 40 - protein, 13 aa, Artificial Sequence
  Synthetic fragment
ACISVDRYLAIVH

SEQ ID NO: 41 - protein, 12 aa, Artificial Sequence
  Synthetic fragment
MATNKDTKIAKK

SEQ ID NO: 42 - protein, 11 aa, Artificial Sequence
  Synthetic fragment
ILIFTDFTCMA

SEQ ID NO: 43 - protein, 17 aa, Artificial Sequence
  Synthetic fragment
KIAKKMAILIFTDFTCM

SEQ ID NO: 44 - protein, 11 aa, Artificial Sequence
  Synthetic fragment
ILIFTDFTCMA

SEQ ID NO: 45 - protein, 11 aa, Artificial Sequence
  Synthetic fragment
KVLSIDYYNMF

SEQ ID NO: 46 - protein, 11 aa, Artificial Sequence
  Synthetic fragment
LMSLDRCLAIC

SEQ ID NO: 47 - protein, 16 aa, Artificial Sequence
  Synthetic fragment
EVKRRALWMVCTVLAV

SEQ ID NO: 48 - protein, 11 aa, Artificial Sequence
  Synthetic fragment
CLFFINTYCSV

SEQ ID NO: 49 - protein, 9 aa, Artificial Sequence
  Synthetic fragment
FCQEEFWGN

SEQ ID NO: 50 - protein, 18 aa, Artificial Sequence
  Synthetic fragment
FCQMRKRRLREQEEFWGN

SEQ ID NO: 51 - protein, 14 aa, Artificial Sequence
  Synthetic fragment
KILLRKFCQIRDHT

SEQ ID NO: 52 - protein, 17 aa, Artificial Sequence
  Synthetic fragment
CHDVLNETLLEGYYAYY

SEQ ID NO: 53 - protein, 11 aa, Artificial Sequence
  Synthetic fragment
YYNHAIDWQTG

SEQ ID NO: 54 - protein, 11 aa, Artificial Sequence
  Synthetic fragment
YAKVSICLPMD

SEQ ID NO: 55 - protein, 11 aa, Artificial Sequence
  Synthetic fragment
ASELSVYTLTV

SEQ ID NO: 56 - protein, 11 aa, Artificial Sequence
  Synthetic fragment
YPLNSCANPFL

SEQ ID NO: 57 - protein, 11 aa, Artificial Sequence
  Synthetic fragment
VAFVIVCCCHV

SEQ ID NO: 58 - protein, 11 aa, Artificial Sequence
  Synthetic fragment
CANPFLYAIFT

SEQ ID NO: 59 - protein, 17 aa, Artificial Sequence
  Synthetic fragment
VRNPQYNPGDKDTKIAK

SEQ ID NO: 60 - protein, 20 aa, Artificial Sequence
  Synthetic fragment
KDTKIAKRMAVLIFTDFICM

SEQ ID NO: 61 - protein, 11 aa, Artificial Sequence
  Synthetic fragment
LAMTLDRHRAI

SEQ ID NO: 62 - protein, 11 aa, Artificial Sequence
  Synthetic fragment
TRNYIHMHLFL

SEQ ID NO: 63 - protein, 11 aa, Artificial Sequence
  Synthetic fragment
KLLKSTLVLMP

SEQ ID NO: 64 - protein, 11 aa, Artificial Sequence
  Synthetic fragment
VFAPVTEEQAR

SEQ ID NO: 65 - protein, 11 aa, Artificial Sequence
  Synthetic fragment
TRNYIHGNLFA

SEQ ID NO: 66 - protein, 11 aa, Artificial Sequence
  Synthetic fragment
RLARSTLTLIP

SEQ ID NO: 67 - protein, 10 aa, Artificial Sequence
  Synthetic fragment
RNYIHMHLFI

SEQ ID NO: 68 - protein, 10 aa, Artificial Sequence
  Synthetic fragment
LARSTLLLIP

SEQ ID NO: 69 - protein, 25 aa, Artificial Sequence
  Synthetic fragment
TLSFVAQNKIDSLNLDEFCNCSEHI

SEQ ID NO: 70 - protein, 11 aa, Artificial Sequence
  Synthetic fragment
PLSAYQIYLGT

SEQ ID NO: 71 - protein, 11 aa, Artificial Sequence
  Synthetic fragment
QSLLVPSIIFI

SEQ ID NO: 72 - protein, 14 aa, Artificial Sequence
  Synthetic fragment
MSFVLVVKLILAIR

SEQ ID NO: 73 - protein, 15 aa, Artificial Sequence
  Synthetic fragment
DSFHILLIMSCQSLL

SEQ ID NO: 74 - protein, 11 aa, Artificial Sequence
  Synthetic fragment
DVRDILHCTNS

SEQ ID NO: 75 - protein, 16 aa, Artificial Sequence
  Synthetic fragment
LIMSCQSLLVPSIIFI

SEQ ID NO: 76 - protein, 376 aa, Homo sapiens
MESPFRGEPGPTCAPSACLPPNSSAWFPGWAEPSNGSAGSEDAQLEPAHISPADPVEITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPVKALDFRTPLKAKIINICIWLLSSSVGISAIVLGGTKVREDVDVIECSLQFPDDDYSWWDLFMKICVFIFAFVIPVLIIIVCYTLMILRLKSVRLLSGSREKDRNLRRITRLVLVVVAVFVVCWTPIHIFILVEALGSTSHSTAALSSYYFCIALGYTNSSLNPILYAFLDENFKRCFRDFCFPLKMRMERQSTSRVRNTVQDPAYLRDIDGMNKPV

SEQ ID NO: 77 - protein, 380 aa, Rattus norvegicus
MESPIQIFRGEPGPTCAPSACLLPNSSSWFPNWAESDSNGSVGSEDQQLEPAHISPAIPVIITAVYSVVFVVGLVGNSLVMFVIIRYTKMKTATNIYIFNLALADALVTTTMPFQSAVYLMNSWPFGDVLCKIVISIDYYNMFTSIFTLTMMSVDRYIAVCHPVKALDFRTPLKAKIINICIWILASSVGISAIVLGGTKVREDVDVIECSLQFPDDEYSWWDLFMKICVFVFAFVIPVLIIIVCYTLMILRLKSVRLLSGSREKDRNLRRITKIVLVVVAVFIICWTPIHIFILVEALGSTSHSTAVLSSYYFCIALGYTNSSLNPVLYAFLDENFKRCFRDFCFPIKMRMERQSTNRVRNTVQDPASMRDVGGMNKPV

SEQ ID NO: 78 - protein, 400 aa, Homo sapiens
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGIPVMFMATTKYRQGSIDCTLTFSHPTWYWENLDKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 79 - protein, 398 aa, Rattus norvegicus
MDSSTGPGNTSDCSDPLAQASCSPAPGSWLNLSHVDGNQSDPCGLNRTGLGGNDSLCPQTGSPSMVTAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIVNVCNWILSSAIGIPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSTRVRQNTREHPSTANTVDRTNHQLENLEAETAPLP

SEQ ID NO: 80 - protein, 372 aa, Homo sapiens
MEPAPSAGAELQPPLFANASDAYPSACPSAGANASGPPGARSASSLALAIAITALYSAVCAVGLLGNVLVMFGIVRYTKMKTATNIYIFNLALADALATSTLPFQSAKYLMETWPFGELLCKAVISIDYYNMFTSIFTLTMMSVDRYIAVCHPVKALDFRTPAKAKIINICIWVLASGVGVPIMVMAVTRPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDIDRRDPLVVAALHLCIALGYANSSLNPVLYAFLDENFKRCFRQLCRKPCGRPDPSSFSRAREATARERVTACTPSDGPGGGAAA

SEQ ID NO: 81 - protein, 359 aa, Rattus norvegicus
MALNSSAEDGIKRIQDDCPKAGRHSYIFVMIPTLYSIIFVVGIFGNSLVVIVIYFYMKLKTVASVFLLNLALADLCFLLTLPLWAVYTAMEYRWPFGNHLCKIASASVTENLYASVFLLTCLSIDRYLAIVHPMKSRLRRTMLVAKVTCIIIWLMAGLASLPAVIHRNVYFIENTNITVCAFHYESRNSTLPIGLGLTKNILGFLFPFLIIITSYTLIWKALKKAYEIQKNKPRNDDIFRIIMAIVLFFFFSWVPHQIFTFLDVLIQLGVIHDCKISDIVDTAMPITICIAYFNNCLNPLFYGFLGKKFKKYFLQLLKYIPPKAKSHSSLSTKMSTLSYRPSDNMSSSAKKPASCFEVE

SEQ ID NO: 82 - protein, 391 aa, Homo sapiens
MFSPWKISMFLSVREDSVPTTASFSADMLNVTLQGPTLNGTFAQSKCPQVEWLGWLNTIQPPFLWVIFVLATLENIFVLSVFCLHKSSCTVAEIYLGNLAAADLILACGLPFWAITISNNFDWLFGETLCRVVNAIISMNLYSSICFLMLVSIDRYLALVKTMSMGRMRGVRWAKLYSLVIWGCTLLLSSPMLVFRTMKEYSDEGHNVTACVISYPSLIWEVFTNMLLNVVGFLLPLSVITFCTMQIMQVLRNNEMQKFKEIQTERRATVLVLVVLLLFIICWLPFQISTFLDTLHRLGILSSCQDERIIDVITQIASFMAYSNSCLNPLVYVIVGKRFRKKSWEVYQGVCQKGGCRSEPIQMENSMGTLRTSISVERQIHKLQDWAGSRQ

SEQ ID NO: 83 - protein, 398 aa, Mus musculus
MDSSAGPGNISDCSDPLAPASCSPAPGSWLNLSHVDGNQSDPCGPNRTGLGGSHSLCPQTGSPSMVTAITIMALYSIVCVVGLFGNFLVMYVIVRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQLENLEAETAPLP

SEQ ID NO: 84 - protein, 401 aa, Bos taurus
MDSGAVPTNASNCIDPFTHPSSCSPAPSPSSWVNFSHLEGNLSDPCGPNRTELGGSDRLCPSAGSPSMITAIIIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDLRTPRNAKIINICNWILSSAIGLPVMFMATTKYRQGSIDCTLTFSHPTWYWENLLKICVFIFAFIMPILIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSTRIRQNTRDHPSTANTVDRTNHQLENLEAETTPLP

SEQ ID NO: 85 - protein, 400 aa, Homo sapiens
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 86 - protein, 400 aa, Sus scrofa
MDSSADPRNASNCTDPFSPSSMCSPVPSPSSWVNFSHLEGNLSDPCIRNRTELGGSDSLCPPTGSPSMVTAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRNGSIDCALTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVYAFLDENFKRCFREFCIPTSSTIEQQNSARIRQNTRDHPSTANTVDRTNHQLENLEAETAPLP

SEQ ID NO: 87 - protein, 383 aa, Homo sapiens
METSGNISDFLYPLSNPVMSNSSVLCRNFSNSTSFLNMNGSSRDSTDEQDKTPVIIAIIITTLYSIVCVVGLVGNVLVMYVIIRYTKMKTATNIYIFNLALADALATSTLPFQSVNYLMGTWPFGDVVCKIVMSIDYYNMFTSIFTLTTMSIDRYIAVCHPVKALDFRTPRNAKIVNVCNWILSSAIGLPVMVMASTTIENQNSPLQVSNFDCTLLFPHPPWYWETLLKICVFILAFIMPVLIITVCYGLMILRLKSVRMLSGSKEKDRNLRRITRMVLVVVAVFIICWTPIHIEVIIKALVTIPNSLFQTVTWHFCIALGYTNSCLNPVLYAFLDENFKRCFREFCVPSPSVLDLQNSTRNSNPQCEGQSSGHKVDRNNRQV